(19) United States
(12) Patent Application Publication
(10) Pub. No.: US 2026/0283657 A1
(43) Pub. Date: Sep. 24, 2026

(54) BONE TRANSPORT IMPLANT

(71) Applicant: NuVasive Specialized Orthopedics, INC., San Diego, CA (US)

(72) Inventors: Emmon Chen, Aliso Viejo, CA (US); Nathan Meyer, Vista, CA (US); Shawn Placie, Aliso Viejo, CA (US); Adam G. Beckett, San Diego, CA (US); Gabriel Buenviaje, Laguna Hills, CA (US); Jason Kwon, Norwalk, CA (US); Alec Broyles, Aliso Viejo, CA (US); Jorge Lopez Camacho, Oxnard, CA (US); Ricky Trieu Quach, Irvine, CA (US); Woong Kim, Aliso Viejo, CA (US); Dalton Jennings, San Diego, CA (US)

(21) Appl. No.: 18/998,023

(22) PCT Filed: Jul. 7, 2023
(86) PCT No.: PCT/US23/27120

§ 371 Date: Jan. 23, 2025

Related U.S. Application Data
(60) Provisional application No. 63/392,201, filed on Jul. 26, 2022.

(51) Int. Cl. A61B 17/72; A61B 17/68; A61B 17/70

(52) U.S. Cl. CPC A61B 17/7216; A61B 17/7016; A61B 2017/681

(57) ABSTRACT

Various implementations include implants and related methods for moving bone. Certain implementations include an implant for moving a plurality of separate bone segments in a patient's body, the implant including: an implantable biocompatible housing; a first shuttle coupled with the housing and defining a first set of one or more holes for receiving a bone anchor coupled to a first bone segment in the plurality of bone segments; a second shuttle coupled with the housing and defining a second set of one or more holes for receiving a bone anchor coupled to a second bone segment in the plurality of bone segments; and a drive system disposed at least partially within the housing and configured to translate the first and second shuttles relative to the housing to move the first and second bone segments, respectively.

### PRIORITY CLAIM

The present patent application claims priority to U.S. Provisional Patent Application No. 63/392,201, filed on Jul. 26, 2022. The foregoing application is incorporated by reference as though fully set forth herein.

### TECHNICAL FIELD

This disclosure generally relates to biocompatible implants. More particularly, the disclosure relates to implants for moving bone in a patient's body.

### BACKGROUND

Implantable bone adjustment systems can beneficially treat a variety of conditions. For example, implantable bone adjustment systems can be used for purposes of distraction osteo-genesis (also known as distraction callotasis and osteodistraction) in applications such as: post osteosarcoma bone cancer; cosmetic lengthening (both legs-femur and/or tibia) in short stature or dwarfism/achondroplasia; lengthening of one limb to match the other (congenital, post-trauma, post-skeletal disorder, prosthetic knee joint), nonunions, etc. Additionally, implantable bone adjustment systems can be used in treatment of various additional conditions and ailments such as scoliosis or osteoarthritis (e.g., knee osteoarthritis).

Example implantable bone adjustment systems are described in U.S. patent application Ser. No. 16/298,339 (filed March 11, 2019), now U.S. Pat. No. 11,439,449; U.S. patent application Ser. No. 13/370,966 (filed February 14, 2011), now U.S. Pat. No. 8,715,282; and U.S. patent application Ser. No. 16/046,909 (filed July 26, 2018), now U.S. Pat. No. 10,918,425, each of which is incorporated herein by reference in its entirety.

### SUMMARY

The needs above, as well as others, are addressed by embodiments of implants and related methods described in this disclosure. All examples and features mentioned below can be combined in any technically possible way.

Various implementations include implants for moving bone in a patient's body, related systems, and methods. Certain implementations include an implant for moving a plurality of bone segments in a patient's body.

In a particular aspect, an implant for moving a plurality of separate bone segments in a patient's body includes: an implantable biocompatible housing defining: a proximal hole for receiving a bone anchor coupled to a proximal bone end-segment; and a distal hole for receiving a bone anchor coupled to a distal bone end-segment; a first shuttle coupled with the housing and defining a first set of one or more holes for receiving a bone anchor coupled to a first bone segment in the plurality of bone segments; a second shuttle coupled with the housing and defining a second set of one or more holes for receiving a bone anchor coupled to a second bone segment in the plurality of bone segments; and a drive system disposed at least partially within the housing and configured to translate the first and second shuttles relative to the housing to move the first and second bone segments, respectively.

In additional particular aspects, a method of performing an acute shortening and lengthening procedure to the plurality of bone segments is performed using an implant described according to aspects of the disclosure.

In further particular aspects, a method of performing a trifocal transport procedure is performed using an implant described according to aspects of the disclosure.

In additional particular aspects, a method of moving a long bone of a patient is performed using an implant described according to aspects of the disclosure. In certain cases, a method of moving at least one of a tibia, a humerus, or a femur of a patient is performed using an implant described according to aspects of the disclosure.

In certain particular aspects, a method includes: implanting an implant in a patient; coupling a proximal portion of the implant to a proximal bone portion; coupling a distal portion of the implant to a distal bone portion; coupling a first shuttle of the implant to a first bone segment between the proximal and distal bone portions; and coupling a second shuttle of the implant to a second bone segment between the proximal and distal bone portions.

In additional aspects, an apparatus includes: a means for causing a first bone segment to move in a first direction at a first rate; and a means for causing the second shuttle to move in a second direction at a second rate.

Implementations may include one of the following features, or any combination thereof.

In some cases, the drive system is configured to cause the first and second shuttles to: move in a same direction at different rates from each other, or move in opposite, converging or diverging directions.

In particular aspects, the first shuttle includes a first translatable rack mounted in the housing and the second shuttle includes a second translatable rack mounted in the housing.

In certain cases, the drive system includes at least one gear, and the implant further includes: a first rail coupled with the first shuttle and the at least one gear; and a second rail coupled with the second shuttle and the at least one gear, where movement of the first rail or the second rail translates movement to the other of the first rail or the second rail.

In some aspects, the drive system directly drives the first shuttle or the second shuttle and indirectly drives the other of the first shuttle or the second shuttle. In certain of these aspects, a single driver drives both the first shuttle and the second shuttle.

In particular implementations, the drive system includes a magnetic driver configured to be driven by an actuator in an external control device from a location external to the patient's body.

In certain cases, the at least one gear of the drive system includes a first gear and a second gear, where the first gear and the second gear are coupled by a common axis. In particular aspects, with the first gear and the second gear coupled by a common axis, the first rail and the second rail are positioned on the same side of the coaxial gears.

In additional particular aspects, the at least one gear of the drive system includes a common gear for driving both the first rail and the second rail, such that the first rail and the second rail are positioned on opposing sides of the common gear.

In certain implementations, the first gear and the second gear have distinct gear ratios.

In particular aspects, the drive system includes at least one of: an electric driver configured to be driven by an actuator in an external control device from a location external to the patient's body, or an ultrasound powered driver configured to be driven by an actuator in an external control device from a location external to the patient's body.

In certain examples, an electric driver can include be driven by a transcutaneous induction power transfer actuator. In additional cases, an ultrasound-powered driver includes an ultrasound-powered piezoelectric driver configured to be driven by an ultrasound actuator.

In some implementations, the implant further includes: a lead screw coupled with the drive system and the first shuttle; a first rack coupled with the first shuttle and the second shuttle; a second rack coupled with the second shuttle and the housing; and a rack gear coupled with the second shuttle, the first rack and the second rack.

In particular aspects, the implant further includes a pinion gear between the rack gear and the second shuttle to modify a gear ratio of the second shuttle relative to the rack gear. In certain cases, the gear ratio can include a ⅔ ratio (or ⅔ rate of movement), a ½ ratio (or ½ rate of movement), a ⅓ ratio (or ⅓ rate of movement).

In some cases, the first rack is fixed to the first shuttle, the second rack is fixed to the housing, and the second shuttle is configured to move relative to the first rack and the second rack.

In certain implementations, the drive system is configured to drive translation of the lead screw, the first shuttle and the first rack, and translation of the first rack engages the rack gear at a first rate and causes translation of the second shuttle at a second, distinct rate.

In particular cases, the drive system includes a first driver coupled with the first shuttle and a second driver coupled with the second shuttle, where the first shuttle and the second shuttle are axially interposed between the first driver and the second driver.

In some aspects, the first driver and the second driver each include a magnetic driver configured to be driven by an actuator in at least one external control device from a location external to the patient's body.

In certain cases, the distinct drivers include separate gear packs for driving the distinct shuttles. In some cases, the first and second drivers can be separately actuated by distinct controllers simultaneously or contemporaneously.

In some cases, the first driver and the second driver are axially separated to mitigate magnetic field interference during actuation by the external control device.

In particular implementations, the first driver and the second driver are configured to be actuated for at least one of converging or series transport of the plurality of bone segments.

In some aspects, the drive system includes a single driver coupled with both the first shuttle and the second shuttle.

In certain cases, the single driver includes a magnetic driver coupled with two distinct gearboxes, where actuation of the magnetic driver causes either symmetrical translation of the first shuttle and the second shuttle or asymmetrical translation of the first shuttle with respect to the second shuttle. In particular aspects, the distinct gearboxes have distinct gear ratios to achieve asymmetrical translation.

In particular aspects, the housing includes a first sub-housing encompassing the first shuttle and a second sub-housing encompassing the second shuttle.

In some implementations, the implant further includes a distraction rod spanning axially between the first sub-housing and the second sub-housing.

In certain aspects, the implant further includes: a first lead screw in the first sub-housing that is coupled with a first portion of the distraction rod; and a second lead screw in the second sub-housing that is coupled with a second portion of the distraction rod, where the drive system includes: a first driver in the first sub-housing that is coupled with the first lead screw; and a second driver in the second sub-housing that is coupled with the second lead screw, where the first driver and the second driver are separately controllable to translate the plurality of bone segments. In certain cases, gear ratios in the gear boxes of the first and second implants can be the same to achieve symmetrical adjustment, or can be distinct to achieve asymmetrical adjustment.

In particular cases, the first lead screw has a first thread orientation and the second lead screw has a second, distinct thread orientation. In certain examples, thread orientations include a first thread orientation such as a left-hand thread orientation and a second thread orientation such as a right-hand thread orientation.

In some aspects, the first driver and the second driver enable a multi-stage lengthening of the plurality of bone segments.

In certain cases, the implant further includes a distraction rod within the housing, where the first shuttle is internal to the distraction rod and the second shuttle is configured to be driven by the distraction rod.

In particular aspects, the drive system is coupled with the distraction rod for driving the distraction rod.

In some cases, the first shuttle includes a first threaded coupling with the distraction rod.

In particular implementations, the drive system includes a lead screw with a second threaded coupling to the distraction rod, where the first threaded coupling and the second threaded coupling have distinct thread pitches. Distinct thread pitches can include for example, a ⅔ pitch, ½ pitch, or ⅓ pitch.

In certain cases, the first threaded coupling has thread pitch that is one-half a thread pitch of the second threaded coupling.

In some aspects, the implant further includes: a distraction rod coupled with the drive system and the first shuttle; and a cable assembly coupled with the drive system and the second shuttle. In particular cases, the drive system includes a gearbox with a connected cable winder.

In particular implementations, the cable assembly includes a winder coupled to the drive system and a cable connected with the second shuttle and the winder, where actuation of the drive system causes translation of the distraction rod and the first shuttle and winding of the winder to translate the second shuttle.

In certain cases, the implant further includes a spring coupled with the second shuttle to provide counter-tension on the second shuttle during the winding of the winder.

In some aspects, the implant further includes a lead screw coupled with the drive system, where each of the first shuttle and the second shuttle is directly coupled with the lead screw.

In particular implementations, the housing includes an axially extending slot enabling direct coupling of the bone anchors to each of the first and second sets of holes.

In certain cases, the first shuttle and the second shuttle are configured to move axially along the slot.

In some aspects, the lead screw includes a first threaded coupling with the first shuttle having a first thread pitch and a second threaded coupling with the second shuttle having a second, distinct thread pitch.

In particular cases, the implant further includes: a lead screw coupled with the drive system; at least one band coupled with the lead screw and each of the first shuttle and the second shuttle; and a reel coupled with the at least one band. In particular cases, a single drive system can drive both shuttles. In some examples, the at least one band includes a polyethylene (PE) band.

In some implementations, the implant further includes a tether on the lead screw that is coupled with the at least one band.

In certain cases, actuation of the drive system causes the lead screw to travel within the housing and cause movement of each of the first shuttle and the second shuttle via the at least one band.

In particular aspects, the housing includes an axially extending slot enabling direct coupling of the bone anchors to each of the first and second sets of holes.

In some implementations, the first shuttle and the second shuttle are configured to move axially along the slot.

In certain aspects, the reel is axially interposed between the first shuttle and the second shuttle.

In particular cases, the implant further includes: a lead screw coupled with the drive system and the first shuttle; and a spring system coupled with the first shuttle and the second shuttle, where the spring system is configured to provide a translation differential between the first shuttle and the second shuttle when driven by the lead screw.

In some cases, a portion of the spring system is axially interposed between the first shuttle and the second shuttle.

In certain implementations, the spring system includes a first spring and a second spring with distinct spring constants, where the first spring is axially interposed between the first shuttle and the second shuttle, and where the second spring is axially interposed between the second shuttle and a distal end of the implant.

In some aspects, when driven by the drive system and the lead screw, the first shuttle translates at a first rate and the second shuttle translates at a second, distinct rate.

In certain cases, the second rate is approximately one-half of the first rate.

In particular implementations, the spring system self-centers the first shuttle and the second shuttle relative to the housing in response to driving by the drive system.

In some cases, the first shuttle includes the drive system having a magnetic driver, a gearbox coupled with the magnetic driver, and a lead screw coupled with the gearbox, where a portion of the lead screw protrudes axially from the first shuttle.

In certain aspects, the magnetic driver is configured to be driven by an actuator in an external control device from a location external to the patient's body, and actuation of the magnetic driver causes the magnetic driver to translate axially within the housing.

In particular implementations, the second shuttle is coupled with the lead screw and is configured to translate with movement of the lead screw.

In some cases, the magnetic driver includes a threaded coupler for modifying a rate of translation of the first shuttle relative to the second shuttle.

In certain cases, the implant further includes a distraction rod housing the drive system, where the first shuttle is coupled with the drive system.

In particular implementations, the drive system includes a magnetic driver and a gearbox for driving a lead screw, and the first shuttle includes an anti-rotation feature for preventing rotation of the first shuttle during rotation of the lead screw.

In certain cases, the second shuttle is coupled with the lead screw and is configured to translate proportionally with the lead screw.

In some aspects, the first shuttle is configured to translate disproportionally with the lead screw.

In particular cases, the first shuttle and the second shuttle each include a housing and the set of one or more holes extend at least partially through the housing.

In certain implementations, the first shuttle and the second shuttle are configured to translate relative to the housing to move the first bone segment and the second bone segment according to a patient adjustment profile for the patient's body. In particular aspects, the patient adjustment profile can be customized for the patient, for example in terms of groove diameter, pitch, and/or grooves per inch/cm.

In some aspects, the implant is configured for intramedullary placement in a patient.

In particular cases, the implant is configured to aid in treatment of a limb length discrepancy or a bone defect in the patient's body.

In certain implementations, the drive system is configured to be powered by an implanted power source.

In some aspects, the implant is configured for extramedullary placement in a patient.

In certain cases, a system includes the implant, wherein the implantable biocompatible housing is configured for intramedullary placement; and an extramedullary plate.

In particular implementations, a method further includes: causing the first shuttle to move in a first direction at a first rate; and causing the second shuttle to move in a second direction at a second rate.

In some cases, the first direction and the second direction are the same direction; and the second rate is greater than the first rate.

In certain aspects, the first direction and the second direction are opposite, converging directions.

In particular cases, the first direction and the second direction are opposite, diverging directions.

In some aspects, causing the first and second shuttles to move occurs simultaneously.

In particular implementations, causing the first and second shuttles to move occurs sequentially.

In certain cases, implanting an implant in a patient includes disposing the implant in an intramedullary manner.

In particular aspects, implanting an implant in a patient includes disposing the implant in an extramedullary manner.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 is a schematic depiction of patient bone segments illustrating modes of bone growth according to various implementations.

FIG. 2 shows a cross-sectional view of an intramedullary implant according to various implementations.

FIG. 3 shows a schematic system diagram of the implant of FIG. 2 according to various implementations.

FIG. 4 is a cross-sectional view of an intramedullary implant according to various additional implementations.

FIG. 5 shows a schematic system diagram of the implant of FIG. 4 according to various implementations.

FIG. 6 is a cross-sectional view of an intramedullary implant according to various implementations.

FIG. 7 shows a schematic system diagram of the implant of FIG. 6 according to various implementations.

FIG. 8 is a cross-sectional view of an intramedullary implant according to various implementations.

FIG. 9 shows a schematic system diagram of the implant of FIG. 8 according to various implementations.

FIG. 10 is a cross-sectional view of an intramedullary implant according to various additional implementations.

FIG. 11 shows a schematic system diagram of the implant of FIG. 10 according to various implementations.

FIG. 12 is a cross-sectional view of an intramedullary implant according to various additional implementations.

FIG. 13 shows a schematic system diagram of the implant of FIG. 12 according to certain implementations.

FIG. 14 is a cross-sectional view of an intramedullary implant according to various implementations.

FIG. 15 shows a schematic system diagram of the implant of FIG. 14 according to various implementations.

FIG. 16 is a cross-sectional view of an intramedullary implant according to various implementations.

FIG. 17 shows a schematic system diagram of the implant of FIG. 16 according to various implementations.

FIG. 18 is a cross-sectional view of an intramedullary implant according to various implementations.

FIG. 19 shows a schematic system diagram of the implant of FIG. 18 according to various additional implementations.

FIG. 20 is a cross-sectional view of an intramedullary implant according to various implementations.

FIG. 21 shows a schematic system diagram of the implant of FIG. 20 according to various implementations.

FIG. 22 is a cross-sectional view of an intramedullary implant according to various implementations.

FIG. 23 shows a schematic system diagram of the implant of FIG. 22 according to various implementations.

FIG. 24 is a cross-sectional view of an intramedullary implant according to various implementations.

FIG. 25 shows a schematic system diagram of the implant of FIG. 24 according to various implementations.

FIG. 26 is a cross-sectional view of an intramedullary implant according to various implementations.

FIG. 27 shows a schematic system diagram of the implant of FIG. 26 according to various implementations.

FIG. 28 illustrates a side view of an example bone transport implant in an example start configuration.

FIG. 29 illustrates a side view of the example bone transport implant of FIG. 28 in an example end configuration.

FIG. 30 illustrates a side view of an example bone transport implant in an example start configuration

FIG. 31 illustrates a side view of the example bone transport implant of FIG. 30 in an example screw exchange configuration.

FIG. 32 illustrates a side view of the example bone transport implant of FIG. 30 in an example end configuration.

FIG. 33 illustrates a side view of an example bone transport implant in an example start configuration.

FIG. 34 illustrates a side view of the example bone transport implant of FIG. 33 in an example screw exchange configuration.

FIG. 35 illustrates a side view of the example bone transport implant of FIG. 33 in an example end configuration.

FIG. 36 illustrates a side view of an example bone transport implant having a first slot rotated relative to a second slot.

FIG. 37 illustrates a simplified proximal-end view of the bone transport implant of FIG. 36.

FIG. 38 illustrates an example bone transport implant having slots angularly offset with respect to each other and which overlap for an overlap zone.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Despite numerous applications, conventional implantable bone adjustment systems are limited in the extent and modality of adjustment. In certain cases, patients can benefit from moving a plurality of separate bone segments, for example, to speed recovery and/or close significant gaps between bone segments. However, conventional approaches require multiple tools, and multiple adjustments. These conventional approaches can be unnecessarily complex, costly, ineffective, or some combination thereof.

This disclosure provides, at least in part, implants for moving a plurality of separate bone segments in a patient's body, and methods that beneficially incorporate such implants to move bone. These implants enable multi-segment adjustment, which can reduce time and complications associated with adjustment procedures. The various disclosed implementations can improve patient outcomes when compared with conventional implantable adjusters. The disclosed implementations can provide adaptability in adjusting bone positioning, enhancing one or both of intraoperative and postoperative engagement with the device. The implant can provide an implantable biocompatible housing with a set of shuttles connected to distinct bone segments, and a drive system that is configured to drive the shuttles to enable movement of the bone segments. In various implementations, the drive system is configured to cause the first and second shuttles to: move in a same direction at different rates from each other, or move in opposite, converging or diverging directions. When compared with conventional approaches, the multi-bone adjustment implants described according to various implementations provide an efficient and simplified mechanism for bone adjustment. The implants described according to various implementations can also reduce health risks for patients when compared with conventional approaches, for example, allowing a single implant to perform functions that might otherwise be performed by multiple implantable devices (with associated implant procedures) or surgical interventions. Further, challenges exist in providing the mechanisms for performing multi-bone adjustment while maintaining a device small enough for useful implantation.

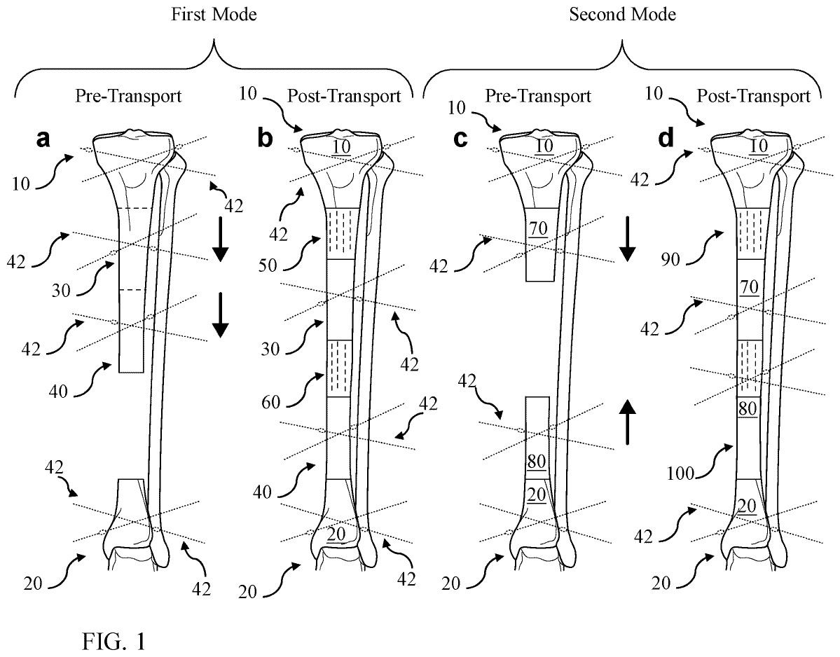

FIG. 1 illustrates two modes of dual-bone (or, multi-bone) transport between bone end-segments 10, 20, according to various implementations. In a first mode, bone segments 30 and 40 are both distracted in a common direction between end-segment 10 and end-segment 20. In certain cases, at least one bone anchor 42 is coupled with a bone segment, and in certain cases, two or more bone anchors 42 are coupled with each bone segment. In this mode, a first bone growth 50 and a second bone growth 60 are formed by distracting bone segments 30 and 40 by moving the bone segments in a same direction (e.g., a same proximal or distal direction), as shown in the post-transport depiction in the first mode of FIG. 1. In a second mode, bone segments 70 and 80 are distracted in a convergent (e.g., by moving the bone segments in opposite, converging directions) approach to form a first bone growth 90 and a second bone growth 100, as shown in the post-transport depiction in the second mode of FIG. 1.

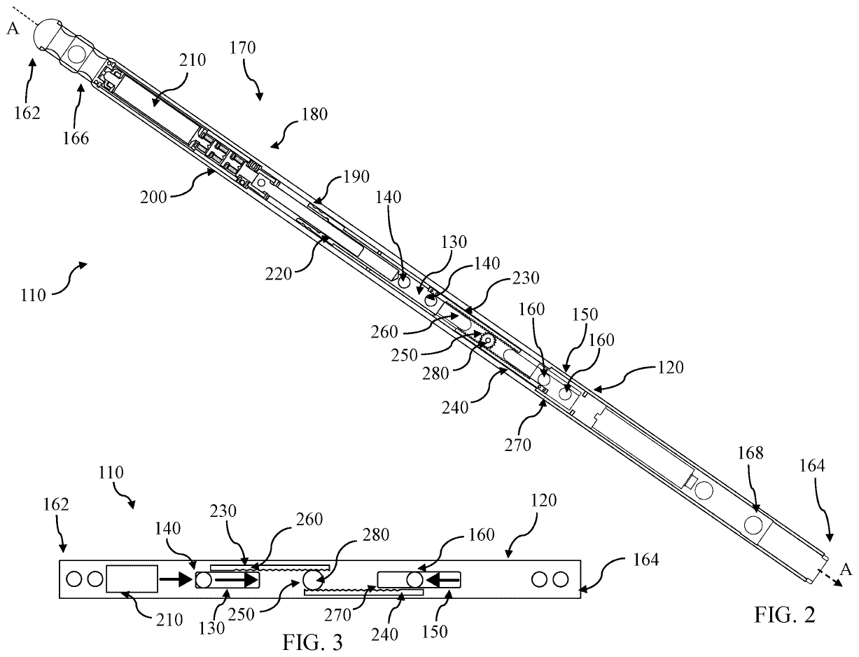

FIG. 2 shows a perspective view of an implant 110 for moving a plurality of separate bone segments in a patient's body, and FIG. 3 is a schematic system diagram illustrating movement of components in the implant 110. In various implementations, the implant 110 (and other implants depicted and described herein) is configured for intramedullary placement in a patient, e.g., to aid in treatment of one or more patient conditions. In certain examples, the implant 110 can be used in a method of intramedullary adjustment of a patient's bone. In a particular example, the implant 110 (and other implant(s) herein) is configured to aid in treatment of a limb length discrepancy and/or a bone defect in the patient's body. In other implementations, the multi-segment transport techniques described herein can be applied in non-intramedullary uses, such as in the correction of scoliosis or for other uses.

In various implementations, the implant 110 includes an implantable biocompatible housing (or, “housing”) 120. A first shuttle 130 is coupled with the housing 120 and defines a first set of one or more holes 140 for receiving a bone anchor 42 (FIG. 1) coupled to a first bone segment (e.g., bone segment 30, FIG. 1). A second shuttle 150 is coupled with the housing 120 and defines a second set of one or more holes 160 for receiving a bone anchor 42 (FIG. 1) that is coupled to a second bone segment (e.g., bone segment 40, FIG. 1).

Spacing of holes 140 relative to the distal end 162 of the implant 110, spacing of holes 160 relative to the proximal end 164 of the implant 110, dimensions of the holes 140, 160, and other dimensional aspects of the holes 140, 160 can be tailored for particular patient parameters and/or treatment profiles. In certain aspects, the bone anchors 42 described herein can include bone screws or other bone fixators or connectors. In various implementations, the distal end 162 and/or proximal end 164 of the implant 110 includes additional holes 166, 168 for receiving bone anchors 42 and providing additional connection points between the implant 110 and the bone segment(s). Examples of bone screws and/or dimensional aspects of the holes 140, 160 are described in U.S. patent application Ser. No. 16/298,339, which is entirely incorporated by reference herein.

The implant 110 can further include a drive system 170 disposed at least partially within the housing 120 and configured to translate the first shuttle 130 and the second shuttle 150 relative to the housing 120 to move the first bone segment and second bone segment, respectively. In particular, the drive system 170 can include a driver 180 coupled with a first adjustment rod 190, where the driver 180 is configured to drive the first adjustment rod 190 to enable translation of the rod 190 relative to the housing 120 (relative to primary axis A). In certain cases, the driver 180 is configured to be actuated by an external control device. In some cases, the external control device includes an external actuator for communicating with the driver 180 from a location external to the patient's body, e.g., a magnetic controller and/or other wireless controller. In certain examples, the driver 180 includes at least one of: an electric driver configured to be driven by an actuator in an external control device from a location external to the patient's body, or an ultrasound powered driver configured to be driven by an actuator in an external control device from a location external to the patient's body. In some cases, an electric driver can be driven by a transcutaneous induction power transfer actuator. In additional cases, an ultrasound-powered driver includes an ultrasound-powered piezoelectric driver configured to be driven by an ultrasound actuator. Various drivers are referred to relative to the implementations described herein. Unless otherwise specified, it is understood that the drivers (e.g., electric, ultrasound, etc.) shown and described can be used to actuate an adjustment rod, lead screw, etc. and cause rotation and/or translation of such components. Additional details of drivers and driven components are not necessarily included in all descriptions of implementations. In particular cases, as noted herein, the drive system 170 is configured to cause the first and second shuttles 130, 150 to: move in a same direction at different rates from each other, or move in opposite, converging or diverging directions.

In particular cases, the driver 180 is configured to control translation of the first adjustment rod 190. In certain implementations, the driver 180 includes a gear module (e.g., with planetary gears) 200 for driving movement of the first adjustment rod 190. In some examples, the gear module 200 is coupled with a magnetic actuator (e.g., permanent magnet) 210 that is rotationally coupled to the gear module 200. However, other driving elements are suitable in place of a magnet. For example, in addition to or instead of the magnet-based driving, one or more of the driving elements can take the form of an implanted electric motor. The implanted electric motor can be powered by an external power source (e.g., via a radiofrequency link, via an inductive connection, or via another technique). The implanted electric motor can be powered by an implanted power source (e.g., a battery, which may be charged by the external power source). The implanted power source may be within the implant (e.g., within a housing thereof) or separate from the implant and coupled to the implant via a cable. In certain cases, the gear module 200 is mounted with radial bearings and/or thrust bearings to a lead screw 220 that drives translation of the first adjustment rod 190.

With reference to the example implant 110 in FIGS. 2 and 3, the first shuttle 130 can include a first translatable rack 230 that is mounted in the housing 120, and a second translatable rack 240 also mounted in the housing 120. In particular cases, the translatable racks 230, 240 are configured to translate along separate sections of the housing 120. In certain examples, the driver 180 (or, drive system) includes at least one gear 250. In these examples, the implant 110 can further include a first rail 260 that is coupled with the first shuttle 130 and the gear(s) 250, and a second rail 270 that is coupled with the second shuttle 150 and the gear(s) 250. In certain cases, movement of the first rail 260 or the second rail 270 translates movement to the other one of the first rail 260 or second rail 270. In certain example implementations, the driver 180 directly drives the first shuttle 130 or the second shuttle 150, and indirectly drives the other one of the first shuttle 130 or the second shuttle 150. In some of these examples, the implant 110 includes a single driver, e.g., driver 180, that is configured to cause translation of both the first shuttle 130 and the second shuttle 150. In these cases, the driver 180 directly drives one of the shuttles and indirectly drives the other one of the shuttles, e.g., via the gear 250. In certain examples, such as certain depictions in FIGS. 2 and 3, the translatable racks 230, 240 are coupled with a single, common gear 250. In such cases, translatable racks 230, 240 can be positioned on opposing sides of the gear 250.

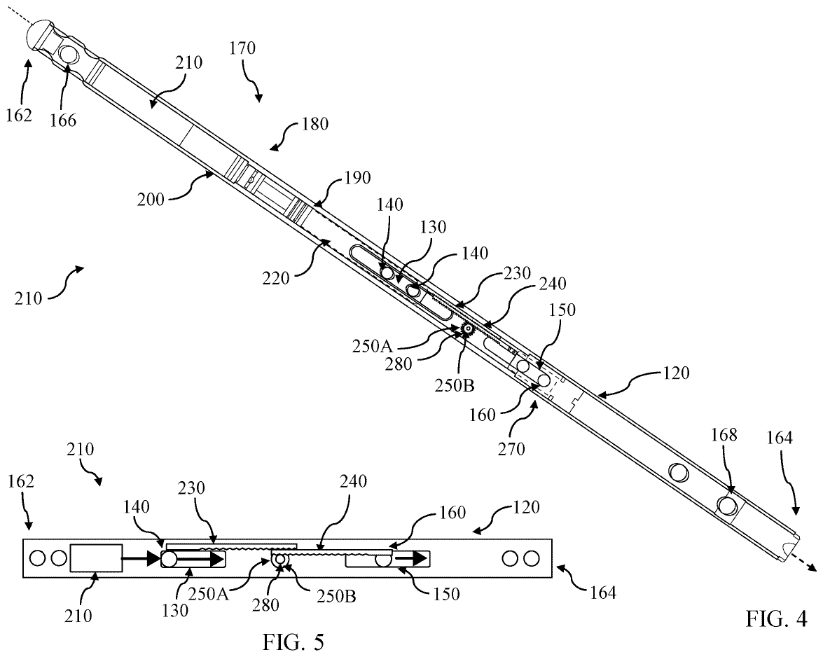

In certain additional examples, the gear(s) 250 include a first gear and a second gear that are coupled by a common axis 280. In FIGS. 2 and 3, the gears 250 can be mounted to the common axis 280 co-axially, such that a first one of the gears 250 is visible in the cross-sectional views while a second (or third, or fourth, etc.) gear is not visible as located behind the visible gear 250. FIGS. 4 and 5 depict another implementation of an implant 210 that includes two distinct gears 250A, 250B, which in certain cases are coaxial, e.g., coupled by a common axis 280. In this example, the gears 250A, 250B have distinct gear ratios (or, sizes) that provide differential translation of movement between the translatable racks 230, 240. In such cases, the translatable racks 230, 240 can be positioned on opposing sides of the gears 250A, 250B, however, in particular implementations with distinct gears 250A, 250B such as illustrated in FIGS. 4 and 5, the translatable racks 230, 240 can be positioned on the same side of gears 250A, 250B. In certain cases, positioning translatable racks 230, 240 on the same side of the gears 250A, 250B can reduce, or mitigate, the overall thickness of the implant 310. In such cases, implanting and/or removing the implant 310 may be more manageable, and imaging of the implant 310 and/or nearby bone may improve.

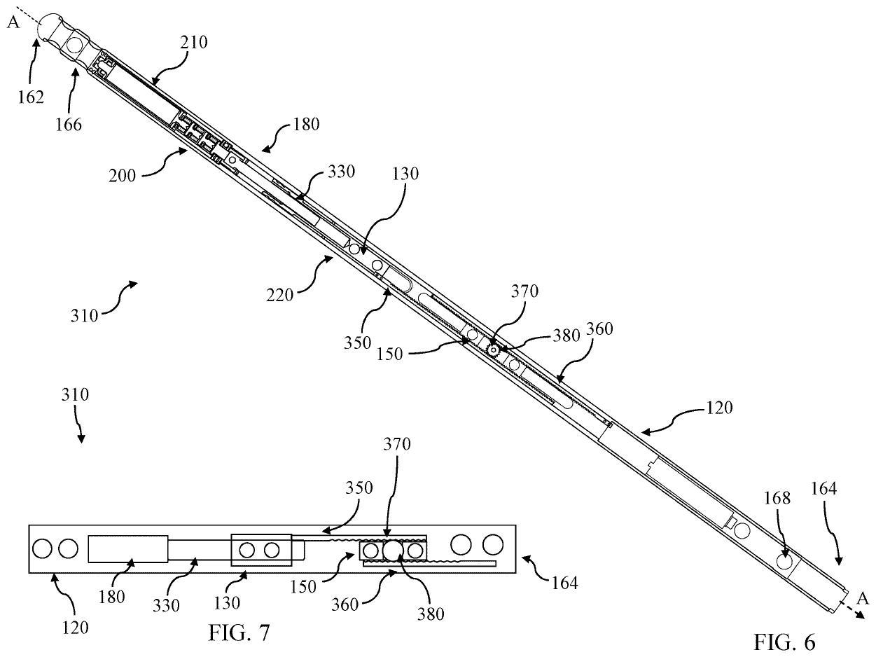

In an additional embodiment, an implant 310 is shown in FIGS. 6 and 7. In these cases, implant 310 includes a lead screw 330 coupled with a drive system (e.g., driver 180) and the first shuttle 130. In certain cases, a first rack 350 is coupled with both the first shuttle 130 and the second shuttle 150. A second rack 360 is coupled with the second shuttle 150 and the housing 120. In this implementation, a rack gear 370 is coupled with the second shuttle 150, the first rack 350 and the second rack 360. In certain cases, a pinion gear 380 is located between (e.g., adjacent) the rack gear 370 and the second shuttle 150 to modify a gear ratio of the second shuttle 150 relative to the rack gear 370. For example, the gear ratio of the pinion gear 380 can be approximately a ⅔ ratio (or ⅔ rate of movement), a ½ ratio (or ½ rate of movement), or a ⅓ ratio (or ⅓ rate of movement). In some aspects, the first rack 350 is coupled with the first shuttle 130 (e.g., fixedly coupled), the second rack 360 is coupled with the housing 120 (e.g., fixedly coupled), and the second shuttle 150 is configured to move relative to the first rack 350 and the second rack 360.

In this example, the driver 180 is configured to drive axial translation of the lead screw 330, and in turn, translate the first shuttle 130 coupled with the lead screw 330 and the first rack 350 coupled with the first shuttle 130. Translation of the first rack 350 engages the rack gear 370 at a first rate, and in turn, translates the second shuttle 150 (along the second rack 360) at a second, distinct rate.

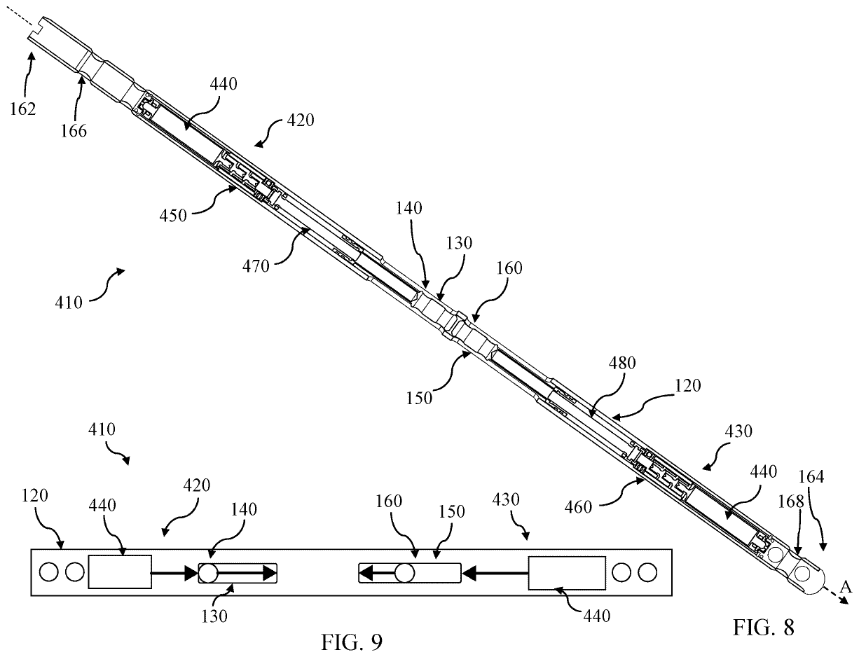

FIGS. 8 and 9 depict an additional implementation of an implant 410 in a multi-driver (or multiple drive system) configuration. In these examples, implant 410 includes a first driver 420 coupled with first shuttle 130, and a second driver 430 coupled with second shuttle 150. In various implementations, the first shuttle 130 and the second shuttle 150 are interposed (e.g., axially interposed) between the first driver 420 and the second driver 430. In certain cases, the first driver 420 and the second driver 430 each include a magnetic driver 440 configured to be driven by an actuator in at least one external control device, e.g., from a location external to the patient's body. In particular aspects, the distinct drivers 420, 430 include separate gear packs 450, 460 for driving the distinct shuttles 130, 150, e.g., via connected lead screws 470, 480, respectively. In such examples, the first driver 420 and second driver 430 can be separately actuated by distinct controllers, e.g., simultaneously or contemporaneously. In certain examples, axial separation of the first driver 420 and the second driver 430 can mitigate magnetic field interference between the drivers 420, 430 during actuation, e.g., by the external control device. According to certain implementations, the first driver 420 and the second driver 430 are configured to be actuated for one or both of converging and series transport of the plurality of bone segments (FIG. 1). In some cases, the first driver 420 and second driver 430 can be actuated at distinct times, e.g., to adjust a position of a first bone segment while maintaining a position of a second bone segment. In additional implementations, the gear packs 450, 460 have distinct gear ratios, which can provide differential adjustment of the first shuttle 130 relative to the second shuttle 150.

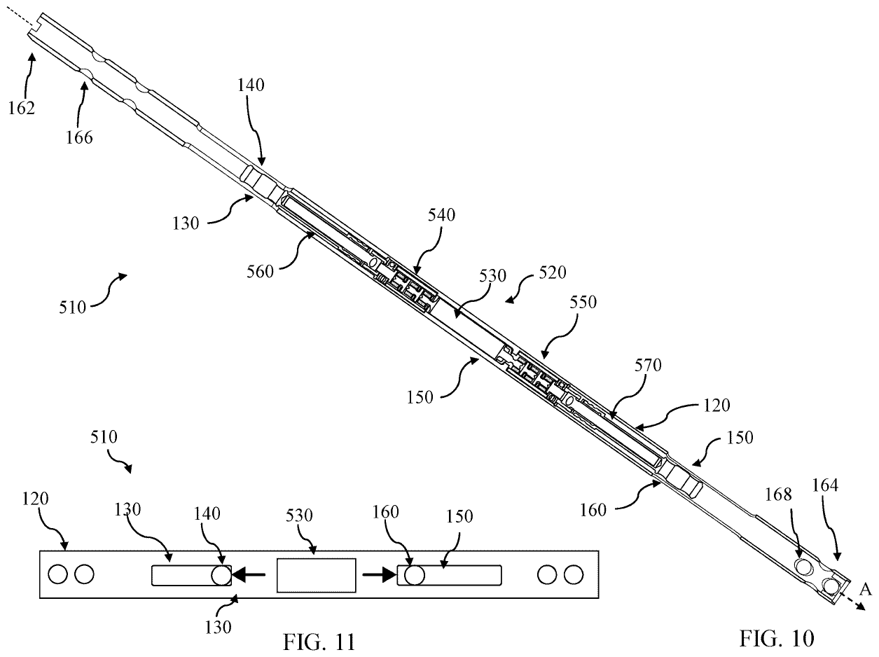

FIGS. 10 and 11 depict an additional implant 510 according to various implementations. As shown, a drive system of implant 510 can include a single driver 520 coupled with both the first shuttle 130 and second shuttle 150 in the housing 120. In certain cases, the single driver 520 is axially interposed between the first shuttle 130 and the second shuttle 150, and is configured to actuate translation of both shuttles 130. In some examples, the single driver 520 includes a magnetic driver 530 coupled with two distinct gear boxes 540, 550 (and corresponding lead screws 560, 570, respectively). In such cases, actuation of the magnetic driver 530 causes either symmetrical translation of the first shuttle 130 and second shuttle 150, or asymmetrical translation of the first shuttle 130 with respect to the second shuttle 150. In certain examples, e.g., where asymmetrical translation is achieved, the distinct gear boxes 540, 550 can have distinct gear ratios. Differential gear ratios can include any gear ratios described herein.

In certain implementations utilizing a single driver to move both shuttles (e.g., in implant 510) enables control of the implant with a single control command (e.g., from an ERC), which can enhance ease of use. In certain implementations utilizing multiple drivers (e.g., in implant 410), separate actuation may be necessary in certain examples. However, the multi-driver configurations can allow for more detail in the adjustment regimen and/or greater differential between bone segments in the adjustment regimen than the single-driver configurations.

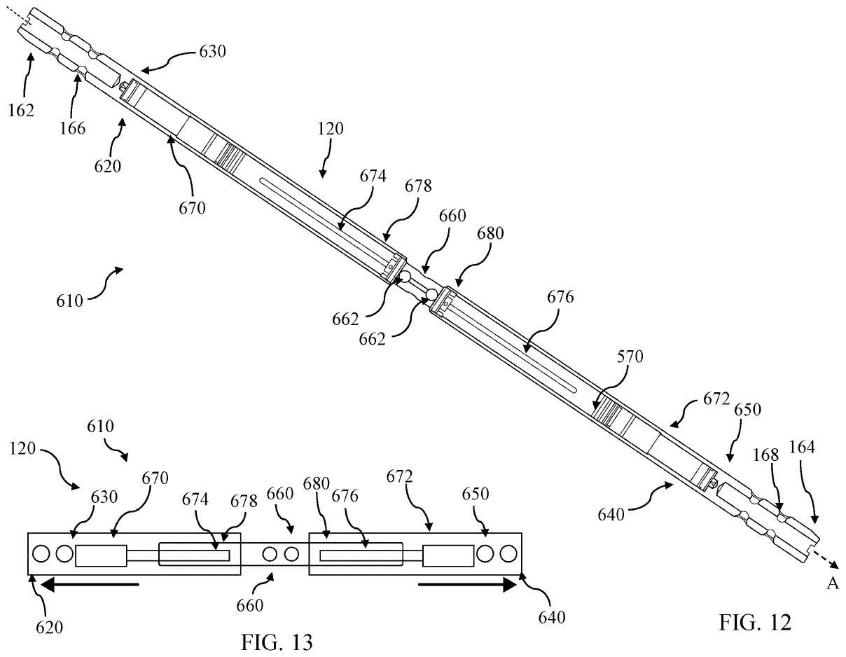

FIGS. 12 and 13 depict an implant 610 according to various additional implementations. In certain aspects, the implant 610 includes a housing 120 with a first sub-housing 620 that encompasses a first shuttle 630 and a second sub-housing 640 that encompasses a second shuttle 650. In these examples, the implant 610 includes a distraction rod 660 that spans axially between the first sub-housing 620 and the second sub-housing 640. According to certain implementations, the drive system in the implant 610 further includes a first drive system (driver) 670 and a second drive system (driver) 672. The implant 610 can further include a first lead screw 674 and a second lead screw 676 (internal to housings 620, 640 in FIG. 12). The first lead screw 674 is located in the first sub-housing 620 and coupled with a first portion 678 of the distraction rod 660. The second lead screw 676 is located in the second sub-housing 640 and is coupled with a second portion 680 of the distraction rod 660. In various implementations, the distraction rod 660 includes a set of one or more holes 662 extending therethrough, e.g., two or more holes for receiving a bone anchor. The first driver 670 in the first sub-housing 620 is coupled with the first lead screw 674, and the second driver 672 in the second sub-housing 640 is coupled with the second lead screw 676. In certain cases, the first driver 670 and the second driver 672 are separately controllable to translate the plurality of bone segments in the patient. According to certain examples, gear boxes in the first and second drivers 670, 672 can have the same gear ratio to achieve symmetrical adjustment of the first shuttle 630 and the second shuttle 650. According to additional examples, gear boxes in the first and second drivers 670, 672 can have distinct gear ratios to achieve asymmetrical adjustment of the first shuttle 630 relative to the second shuttle 650. In certain examples, the first lead screw 674 has a first thread orientation (e.g., left-hand thread orientation) and the second lead screw 676 has a second, distinct thread orientation (e.g., right-hand thread orientation). In particular cases, the first driver 670 and the second driver 672 enable a multi-stage lengthening of the plurality of bone segments, for example, by actuating a first one of the drivers and subsequently actuating a second one of the drivers (or both drivers).

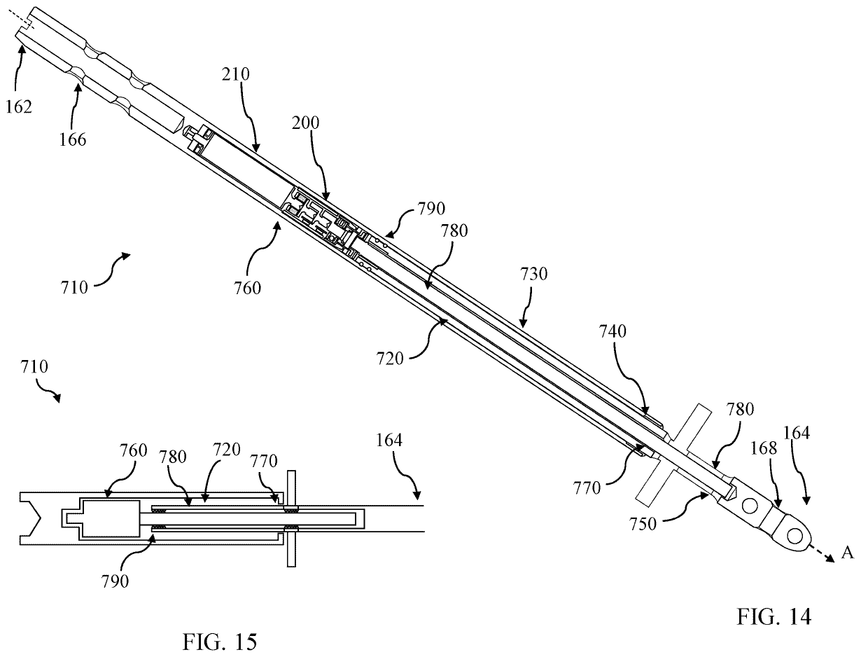

FIGS. 14 and 15 illustrate an implant 710 according to additional implementations. In these examples, implant 710 can include a distraction rod 720 within a housing 730. In such examples, a first shuttle 740 is internal to the distraction rod 720, and a second shuttle 750 is configured to be driven by the distraction rod 720. In particular aspects, a drive system (driver) 760, similar to other drive systems depicted herein, is coupled with the distraction rod 720 and configured to drive the distraction rod 720. In some cases, as illustrated in FIG. 15, the first shuttle 740 includes a first threaded coupling 770 with the distraction rod 720. As is further shown in FIGS. 14 and 15, the driver 760 includes a lead screw 780 with a second threaded coupling 790 to the distraction rod 720. In various implementations, the first threaded coupling 770 and the second threaded coupling 790 have distinct thread pitches. In one example, the first threaded coupling 770 has a thread pitch that is one-half of a thread pitch of the second threaded coupling 790. In additional examples, the first threaded coupling 770 has a thread pitch that is two-thirds or one-third of a thread pitch of the second threaded coupling 790. In various implementations, the internal shuttle 740 enables a single distraction rod (and single driver 760) to control adjustment of both shuttles 740, 750 (and consequently, multiple bone segments). In certain cases, differential thread pitches between the couplings 770, 790 can enable a single driver 760 to control differential adjustment of the first shuttle 740 as compared with the second shuttle 750, e.g., enabling differential adjustment of multiple bone segments.

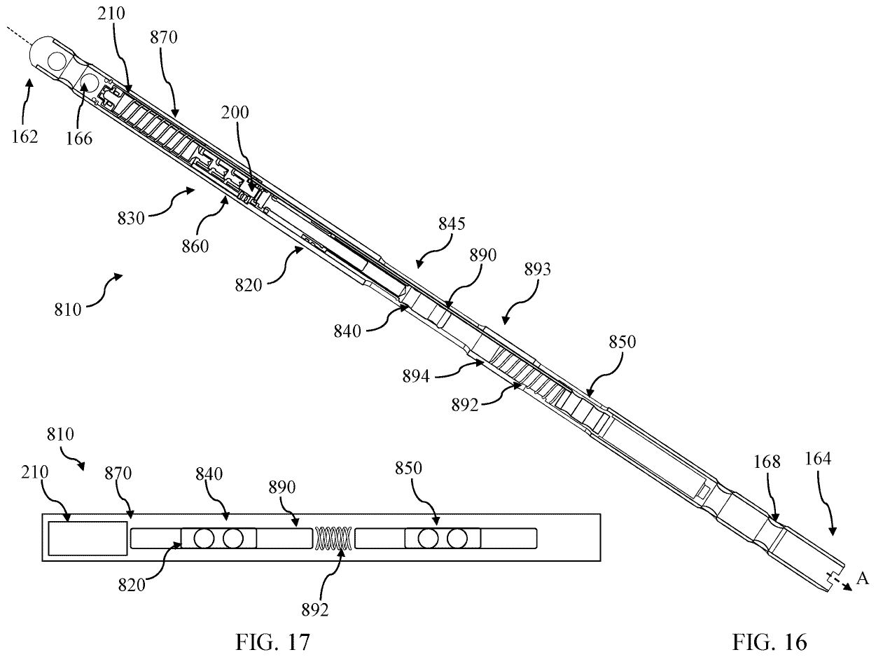

FIGS. 16 and 17 illustrate an implant 810 according to various additional implementations. In some aspects, the implant 810 includes a distraction rod 820 coupled with a driver 830 and a first shuttle 840. The implant 810 can further include a cable assembly 845 coupled with the driver 830 and a second shuttle 850. In particular cases, the driver 830 includes a gearbox 860 with a connected cable winder 870. In particular implementations, the winder 870 is coupled to the driver 830 and a cable 890 is connected with the second shuttle 850 and the winder 870. The cable 890 can be pinned, fastened, bolted, or otherwise connected to the second shuttle 850 and the winder 870. During use, actuation of the driver 830 causes translation of the distraction rod 820 and the first shuttle 840, and also causes winding of the winder 870 to translate the second shuttle 850. In additional examples, the implant 810 further includes a spring 892 coupled with the second shuttle 850 to provide counter-tension on the second shuttle 850 during winding of the winder 880. In various implementations, the spring 892 is fixed to the housing 893 on a side 894 that opposes the side connected with the second shuttle 850. In such cases, the spring constant of the spring 892 and the winder 880 can be selected or otherwise calibrated to control the amount of counter-tension on the second shuttle 850 during winding of the winder 880.

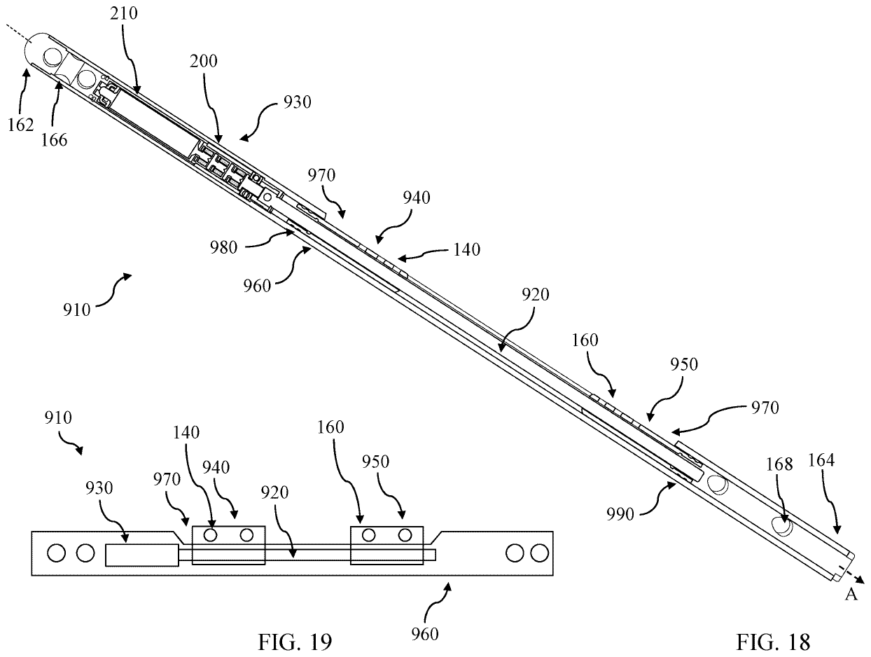

FIGS. 18 and 19 illustrate an implant 910 according to various implementations. As shown, the implant 910 can include a lead screw 920 coupled with a drive system (driver) 930. In these implementations, two shuttles, including a first shuttle 940 and a second shuttle 950, are each directly coupled with the lead screw 920. In certain cases, the lead screw 920 passes through a slot in the shuttles 940, 950, or is otherwise directly coupled with the shuttles 940, 950 via a fastener, pin, clip, threaded connection, etc. In various implementations, a housing 960 for the implant 910 includes an axially extending slot 970 that enables direct coupling of the bone anchors (FIG. 1) to each of the first and second sets of holes 140, 160. In these cases, the holes 140, 160 are directly accessible to the bone anchors via the slot 970, e.g., without passing through the housing 960. According to some examples, the slot 970 extends along one side of the housing 960, and the holes 140, 160 enable the bone anchors to pass therethrough without passing through the housing 960. In various implementations, the first shuttle 940 and the second shuttle 950 are configured to move axially along the slot 970, e.g., as the lead screw 920 translates relative to the housing 960. In particular implementations, the lead screw 920 includes at least two distinct threaded couplings that enable differential adjustment of the bone segments. For example, the lead screw 920 can include a first threaded coupling 980 that is coupled with the first shuttle 940 and a second threaded coupling 990 that is coupled with the second shuttle 950. In various implementations, the first threaded coupling 980 has a first thread pitch and the second threaded coupling has a second, distinct thread pitch. In various implementations, the difference in thread pitch between the first and second threaded couplings 980, 990 can be similar to other examples described herein, e.g., two-thirds, one-half, one-third, etc. In particular cases, different thread pitches between the couplings 980, 990 enables differential axial adjustment of the first shuttle 940 relative to the second shuttle 950 when driven by the lead screw 920.

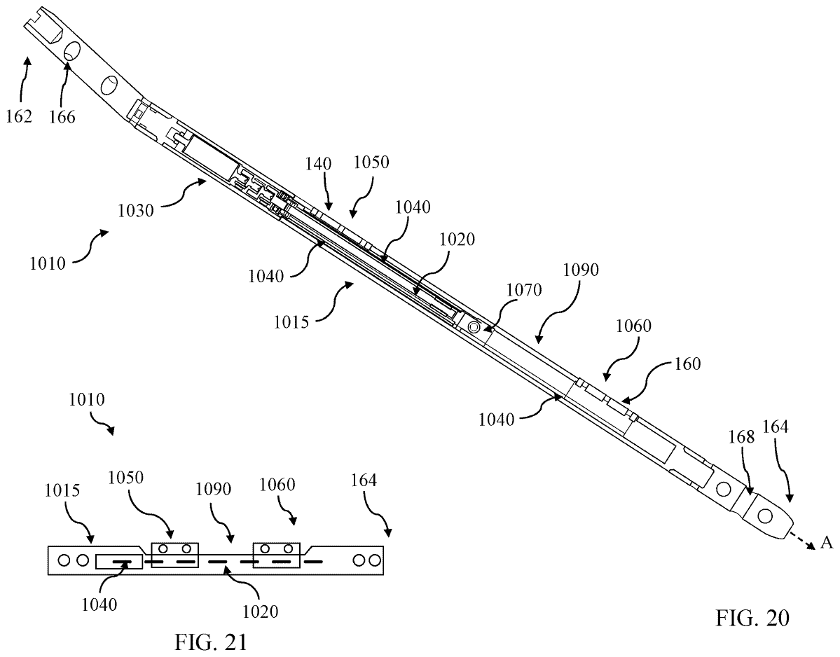

FIGS. 20 and 21 illustrate an implant 1010 according to various implementations. In these cases, the implant 1010 includes a housing 1015 with a lead screw 1020 coupled with a drive system (driver) 1030, and one or more band(s) 1040 coupled with the lead screw 1020 and each of a first shuttle 1050 and a second shuttle 1060. In some examples, the band(s) 1040 includes a polyethylene band. The implant 1010 further includes a reel 1070 coupled with the band(s) 1040, e.g., to reel and un-reel the band(s) 1040 during use. In additional implementations, a tether 1080 is coupled to the lead screw 1020 and the band(s) 1040, for example, to tether the band(s) 1040 to the lead screw 1020. In particular examples, the tether 1080 is mounted to an end of the lead screw 1020. In certain examples, a single driver, e.g., driver 1030, can drive both the first shuttle 1050 and the second shuttle 1060. That is, actuation of the driver 1030 causes the lead screw 1020 to travel within the housing 1015 and cause movement (via band(s) 1040)) of each of the first shuttle 1050 and the second shuttle 1060. In certain examples, the first shuttle 1050 is directly coupled to the lead screw 1020, and the second shuttle 1060 is indirectly coupled to the lead screw via the band(s) 1040 and tether 1080. In particular aspects, the housing 1015 includes an axially extending slot 1090 enabling direct coupling of the bone anchors (FIG. 1) to each of the first and second sets of holes 140, 160 in respective shuttles 1050, 1060, e.g., without passing through housing 1015. In some implementations, similar to implant 910 in FIGS. 18 and 19, the first shuttle 1050 and the second shuttle 1060 are configured to move axially along the slot 1090. In certain aspects, the reel 1070 is axially interposed between the first shuttle 1050 and the second shuttle 1060. In these cases, the first shuttle 1050 and the second shuttle 1060 are configured to translate within separate slots in the housing 1015, e.g., where the slots include axial ends or stops for limiting translation of the shuttles 1050, 1060.

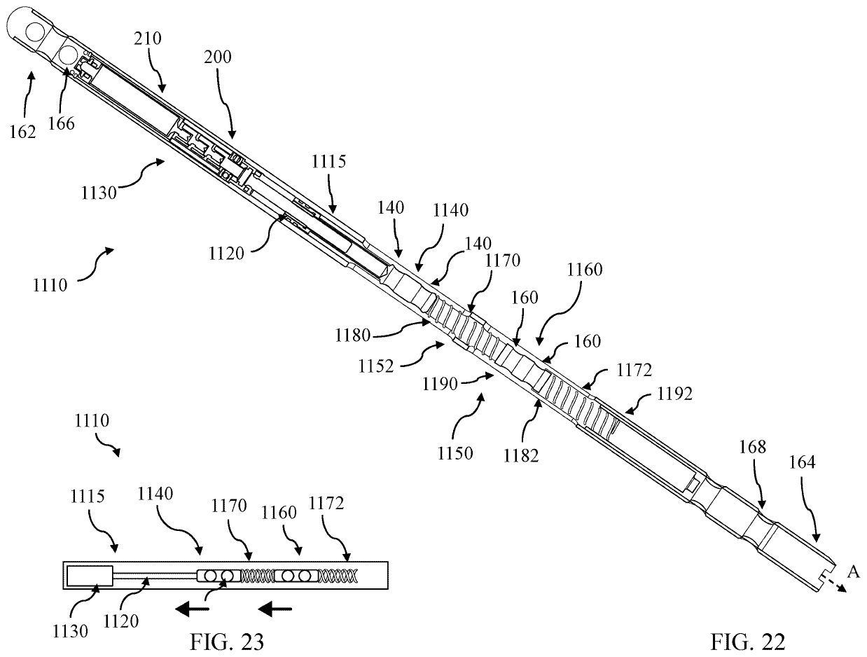

FIGS. 22 and 23 illustrate an implant 1110 according to various additional implementations. As shown, implant 1110 includes a housing 1115 that can include a lead screw 1120 coupled with a first shuttle 1140 and a drive system 1130 for driving the lead screw 1120 (e.g., according to various implementations herein). A spring system 1150 is coupled with the first shuttle 1140 and a second shuttle 1160. In various implementations, the spring system 1150 is configured to provide a translational differential between the first shuttle 1140 and the second shuttle 1160 when the shuttles 1140, 1160 are driven by the lead screw 1120. In one example configuration depicted in FIGS. 22 and 23, the spring system 1150 includes a first portion 1152 that is axially interposed between the first shuttle 1140 and the second shuttle 1160. In certain cases, the spring system 1150 includes a first spring 1170 coupled with a distal end 1180 of the first shuttle 1140 and a proximal end 1190 of the second shuttle 1160. In some of these cases, the spring system 1150 can include a second spring 1172 coupled with a distal end 1182 of the second shuttle 1160. In these examples, the first spring 1170 is axially interposed between the first shuttle 1140 and the second shuttle 1160. In particular implementations, the first spring 1170 has a first spring constant and the second springs 1172 has a second, distinct spring constant. In certain examples, as noted herein, the first spring 1170 is axially interposed between the first shuttle 1140 and the second shuttle 1160, and the second spring 1172 is axially interposed between the second shuttle 1160 and a distal end 164 of the implant 1110. In certain cases, the second spring 1172 is fixed (e.g., fastened, pinned, integrally formed, etc.) at its distal end 1192 to a portion of the housing 1115. In certain cases, when driven by the drive system 1130 and the lead screw 1120, the first shuttle 1140 translates at a first rate and the second shuttle 1160 translates at a second, distinct rate. In particular examples, the second rate is approximately one-half of the first rate. In further particular examples, the spring system 1150 is configured to center (or, “self-center”) the first shuttle 1140 and the second shuttle 1160 relative to the housing 1115 in response to driving by the drive system 1130.

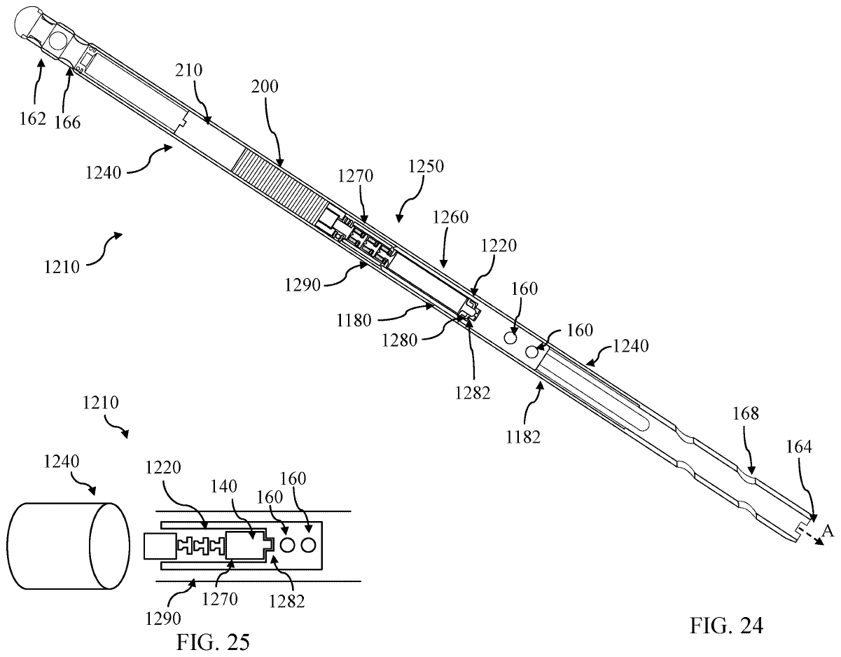

FIGS. 24 and 25 illustrate an implant 1210 according to additional implementations. In these cases, the implant 1210 has a first shuttle 1220 coupled with a housing 1240. In these implementations, the first shuttle 1220 includes a drive system 1250 with a magnetic driver 1260, and a gearbox 1270 coupled with the driver 1260. In certain implementations, a stem 1280 protrudes axially from the magnetic driver 1260 and is at least partially surrounded by a radial bearing 1282, e.g., for maintaining axial alignment of the magnetic driver 1260 and mitigating friction during movement. In some cases, as described herein, the magnetic driver 1260 is configured to be driven by an actuator in an external control device, e.g., to cause the driver 1260 to translate axially within the housing 1240. In some cases, the magnetic driver 1260 includes a threaded coupler 1290 for controlling a rate of translation of the first shuttle 1220 relative to the housing 1240. In certain cases, the implant 1210 can include a second self-driven shuttle (not shown), similar to the first shuttle 1220 and opposing the first shuttle 1220 in the housing 1240 (e.g., similar to FIGS. 8 and 9). In these implementations, the second self-driven shuttle can include a drive system (similar to drive system 1250) with a magnetic driver with an axially extending stem (similar to magnetic driver 1260), and a gearbox (similar to gearbox 1270) coupled with the driver. In various implementations, the magnetic driver in the second self-driven shuttle is configured to be driven by an actuator in an external control device, e.g., to cause the second self-driven shuttle to translate axially within the housing 1240. In certain cases, the magnetic driver in the second self-driven shuttle includes a second threaded coupler for controlling a rate of translation of the second self-driven shuttle relative to the housing 1240. In some cases, the threaded couplers have differential thread configurations (e.g., different thread pitch and/or orientation) to provide a differential translation rate for the first shuttle 1220 relative to the second (self-driven) shuttle. In various implementations, the shuttle(s) are configured to translate axially along a set of internal guides (or rails). Movement of the first and second shuttles (and corresponding distraction rods) can be axially limited by at least one stop, e.g., an axially interposed stop such as a partition or a wall, similar to configurations shown in FIGS. 8 and 9.

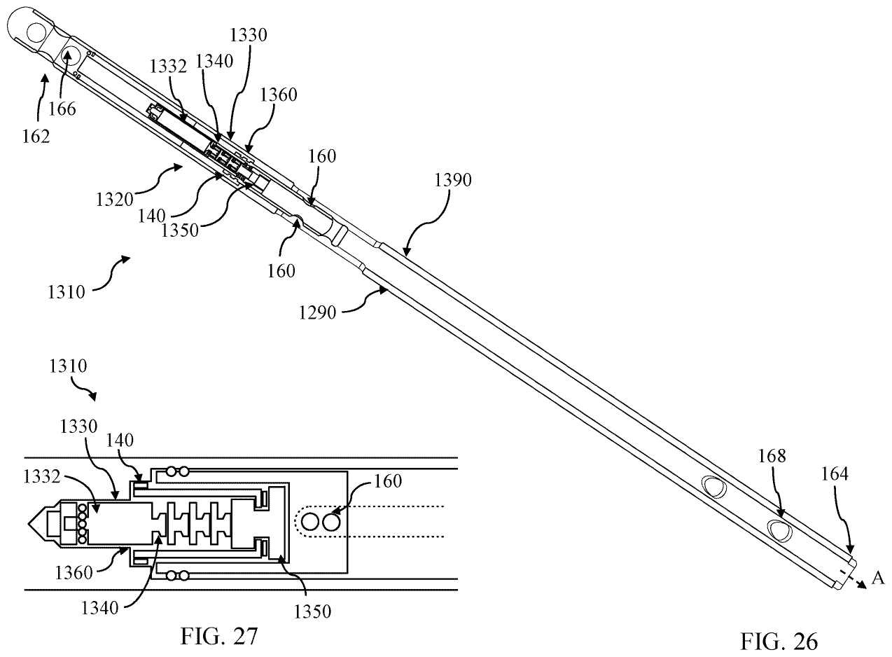

FIGS. 26 and 27 illustrate an implant 1310 according to additional implementations. In these cases, the implant 1310 includes a drive system 1320 that is housed within a distraction rod 1330. In particular examples, an entirety of the drive system 1320 is housed within the distraction rod 1330, although this is not necessarily true in all implementations. The drive system 1320 can include a magnetic driver 1332 and a gearbox 1340 for driving a lead screw 1350. In these cases, a first shuttle 1360 is coupled with the drive system 1320 and the lead screw 1350. In operation, the magnetic driver 1330 is actuatable, e.g., via a remote actuator and/or an on-board actuator, to actuate the gearbox 1340 and in turn drive the lead screw 1350. In certain cases, the first shuttle 1360 includes an anti-rotation feature such as a set of complementary tabs/slots, flat surfaces (flats), etc., for preventing rotation of the first shuttle 1360 during rotation of the lead screw 1350. In some such cases, the first shuttle 1360 is configured to translate disproportionally with the lead screw 1350 relative to housing 1390. For example, the anti-rotation feature can prevent rotation of the first shuttle 1360 during (at least a portion of the) rotation of the lead screw 1350. In such cases, the anti-rotation feature can extend axially along a portion of the length (axial length along axis A) of the housing 1390. In certain cases, the implant 1310 can include a second shuttle (not shown), similar to the first shuttle 1360 and opposing the first shuttle 1360 in the housing 1390 (e.g., similar to FIGS. 8 and 9). In these implementations, the second shuttle can include a drive system (similar to drive system 1320) with a magnetic driver and a gearbox that is either housed within a second distraction rod (similar to distraction rod 1330). In various implementations, the magnetic driver in the second shuttle is configured to be driven by an actuator in an external control device, e.g., to cause the second shuttle to drive the second lead screw (and corresponding second distraction rod) axially within the housing 1390. In certain cases, first and second shuttles include distinct magnetic drivers. In some cases, the second shuttle includes an anti-rotation feature such as a set of complementary tabs and slots, flat surfaces (flats), etc., for preventing rotation of the second shuttle during rotation of the second lead screw (similar to first shuttle 1360). Movement of the first and second shuttles (and corresponding distraction rods) can be axially limited by at least one stop, e.g., an axially interposed stop such as a partition or a wall, similar to configurations shown in FIGS. 8 and 9.

In traditional bone transport implants, a screw exchange procedure can be used to permit transport of bone carried by a single shuttle across an implant's bridge that divides the complete transport length across two separate slots. To use the complete length of the two slots combined, there are two locking screw options on the implant which require a screw exchange procedure to provide continuous transport across the entire length of the slot. The procedure is as follows: the first screw arrives at the end of the first slot, the second screw option is accessible within the second slot on the other side of the support bridge, and is placed before removing the first screw. The first screw is removed, clearing the support bridge, and the transport continues uninterrupted until the second screw reaches the end of the second slot. While this screw exchange procedure can permit the implant to have a bridge that strengthens the implant, it requires the patient to undergo a new operation to perform the screw exchange.

An example benefit of the transport of a plurality of bone segments is that the intramedullary implant can include a plurality of bone transport slots separated by one or more bridges of material without requiring a screw exchange procedure for full bone transport coverage. By dividing the entire slot in which the bone transport shuttles run into multiple slots, the resulting supporting bridge between the slots increases the strength of the implant.

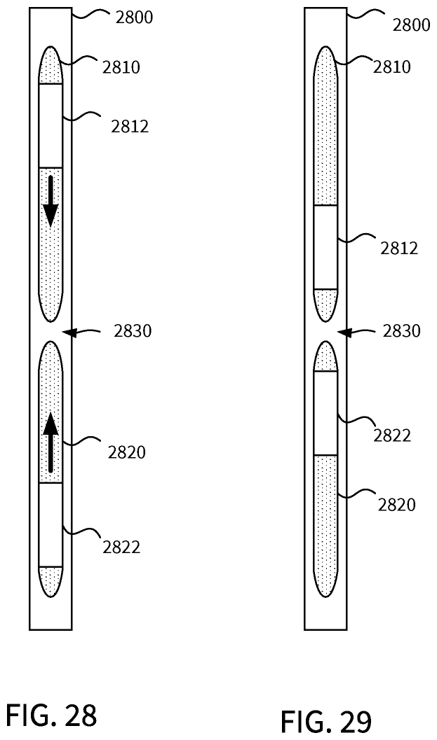

FIGS. 28 and 29 illustrate a first example bone transport implant 2800 in respective start and end configurations. The start configuration can be a configuration that the implant 2800 is in before, during, or after implantation prior to significant bone transport. The end configuration can be a configuration that the implant 2800 after sufficient actuation that significant bone transport has taken place (e.g., after sufficient bone transport has taken place that a bone transport treatment plan for the recipient of the implant has bene satisfied). The bone transport implant 2800 includes a first longitudinal slot 2810 in which a first bone transport shuttle 2812 is disposed and a second longitudinal slot 2820 in which a second bone transport shuttle 2822 is disposed.

A bridge 2830 of material separates the first longitudinal slot 2810 from the second longitudinal slot 2820. The bridge 2830 can be any material or structure that separates the slots 2810, 2820. In an example, the bridge 2830 can be housing material of the implant 2800. In some examples, the bridge 2830 is disposed such that one or more of the shuttles 2812, 2822 can pass underneath the bridge 2380. However, a bone screw or other structure linking a respective shuttle 2812, 2822 to a respective bone segment may block movement of a connected shuttle 2812, 2822 under the bridge 2380. However, in the configuration of this implant 2800, because a screw exchange procedure is not needed to cover bone movement for a full length of the slots, the bridge 2830 in this example may be configured such that a shuttle 2812, 2822 cannot pass underneath.

In the illustrated configuration, the bone transport shuttles 2812, 2822 begin near opposite ends of the implant 2800 and are configured to converge via actuation of the implant (e.g., as described in more detail elsewhere herein). As shown in FIG. 29, the shuttles 2812, 2822 can converge near the bridge 2830. In other words, the respective bone segments carried by the shuttles 2812, 2822 can meet at the bridge 2830 (e.g., the portions of the bone segments touch at a location that overlaps the bridge 2830).

In this example, by having at least two bone segments moving toward the bridge 2830, the implant 2800 is able to include a bridge 2830 to increase strength without needing the recipient of the implant to undergo a screw exchange procedure to obtain the full range of transport.

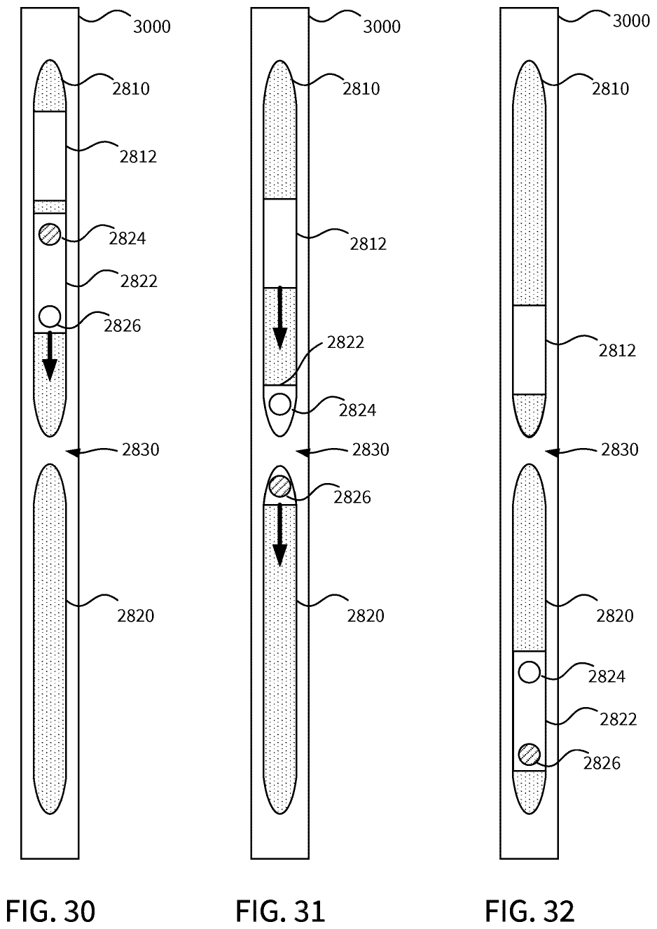

Nonetheless, an implant having a plurality of shuttles may benefit from being supporting a screw exchange procedure, such as is shown in FIGS. 30-32.

FIG. 30 illustrates a second example bone transport implant 3000 in an example start configuration (e.g., in an initially implanted configuration). Here, the implant 3000 is similar to the implant 2800 but with the shuttles 2812, 2822 starting in the same slot 2810 and being configured to move in a same direction (e.g., using any such techniques described elsewhere herein). The second slot 2820 starts (e.g., is implanted) without a shuttle disposed within. The second shuttle 2822 includes at least a proximal anchor 2824 and a distal anchor 2826 for coupling to the bone segment. The anchors 2824, 2826 can be, for example, screw holes into which screws can be secured. The proximal anchor 2824 can be coupled to the bone segment during implantation and the distal anchor 2826 can be unused to start (e.g., be unconnected from a bone segment).

FIG. 31 illustrates the second simplified bone transport implant 3000 in an example screw exchange configuration. The illustrated configuration can be achieved after sufficient actuation of the implant 3000 (e.g., using any technique described herein). For instance, the actuation can cause the second shuttle 2822 to be disposed such that the bridge 2830 is between the proximal anchor 2824 and the distal anchor 2826. Using the screw exchange procedure described above or elsewhere, the distal anchor 2826 can be coupled to the bone segment (e.g., with a screw) and the proximal anchor 2824 can be decoupled from the bone segment. Thus, following the screw exchange procedure, the shuttle 2822 can proceed past the bridge without the bone anchor blocking movement of the shuttle. Meanwhile, actuation can cause the first shuttle 2812 to proceed about halfway along the first slot 2810. Upon continued actuation, the implant 3000 can arrive at the configuration shown in FIG. 32.

FIG. 32 illustrates the second simplified bone transport implant 3000 in an example end configuration. As illustrated, the first and second shuttles 2812, 2822 arrive at the respective distal ends of their respective first and second slots 2810, 2820.

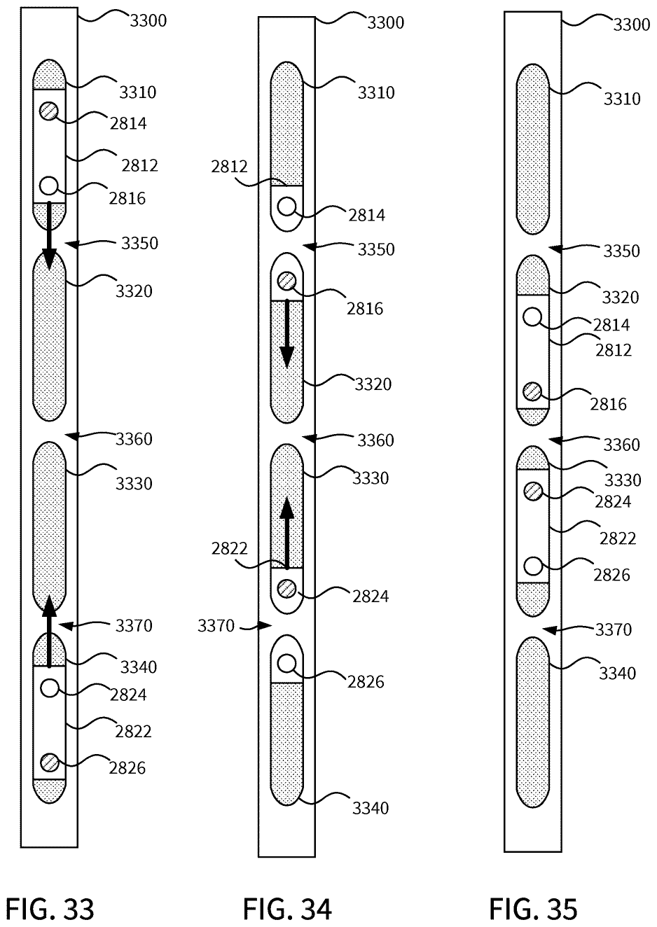

In the illustrated configuration of FIGS. 30-32, the first shuttle 2812 travels only within the first slot 2812 and does not cross the bridge 2830. The second shuttle 2822 is the only shuttle that crosses the bridge 2830. However, an implant need not be so configured. For instance, FIGS. 33-35 show an implant 3300 configured such that all shuttles start and end in different slots.

FIG. 33 illustrates the implant 3300 in an example start configuration (e.g., the configuration of the implant 3300 as implanted). Here, the implant 3300 includes a first slot 3310, a second slot 3320, a third slot 3330, and a fourth slot 3340. Between the first slot 3310 and the second slot 3320 is a first bridge 3350. Between the second slot 3320 and the third slot 3330 is a second bridge 3360. Between the third slot 3330 and the fourth slot 3340 is a third bridge 3370. A first shuttle 2812 having a proximal anchor 2814 and a distal anchor 2816 are disposed within the first slot 3310. A first bone segment can be coupled to the first shuttle 2812 via the proximal anchor 2814. The distal anchor 2816 is not connected to the first bone segment. A second shuttle 2822 having a proximal anchor 2824 and a distal anchor 2826 is disposed in the fourth slot 3340. A second bone segment can be coupled to the second shuttle 2822 via the distal anchor 2826. The proximal anchor 2824 is not connected to the second bone segment. Thus, the first slot 3310 (e.g., the most proximal slot) and the fourth slot 3340 (e.g., the most distal slot) include respective shuttles 2812 and 2822, and the second slot 3320 and the third slot 3330 lack a shuttle. The implant 3000 can be configured (e.g., using any technique described herein) such that the shuttles 2812, 2822 can move toward each other, eventually passing under respective bridges and arriving at the middle two slots 3320, 3330.

FIG. 34 illustrates the third example bone transport implant 3300 in an example screw exchange configuration in which at least a portion of the first shuttle 2812 overlaps the first bridge 3350 (e.g., are both intersected by a same plane perpendicular to a length of the implant 3300) and at least a portion of the second shuttle 2822 overlaps the third bridge 3370. The illustrated configuration can be achieved after sufficient actuation of the implant 3300. For instance, the actuation can cause the first shuttle 2812 to be disposed such that the bridge 3350 is disposed between the proximal anchor 2814 and the distal anchor 2816. The actuation can further cause the second shuttle 2822 to be disposed such that the bridge 3370 is disposed between the proximal anchor 2824 and the distal anchor 2826.

Using the screw exchange procedure described above or elsewhere, the distal anchor 2816 of the first shuttle 2812 can be coupled to the first bone segment (e.g., with a screw) and the proximal anchor 2814 of the first shuttle 2812 can be decoupled from the bone segment. During the screw exchange procedure, the proximal anchor 2824 of the second shuttle 2822 can be coupled to the second bone segment (e.g., with a screw) and the distal anchor 2826 of the second shuttle 2822 can be decoupled from the second bone segment. Thus, following the screw exchange procedure, both shuttles 2812, 2822 can proceed past the respective bridges 3350, 3370 without being blocked (e.g., by a bridge being hit by screw connecting a bone segment to an anchor). Upon continued actuation, the implant 3000 can arrive at the configuration shown in FIG. 35.

FIG. 35 illustrates the third simplified bone transport implant 3300 in an example end configuration. As illustrated, the first and second shuttles 2812, 2822 arrive at the respective distal and proximal ends of their respective second and third slots 3320, 3330.

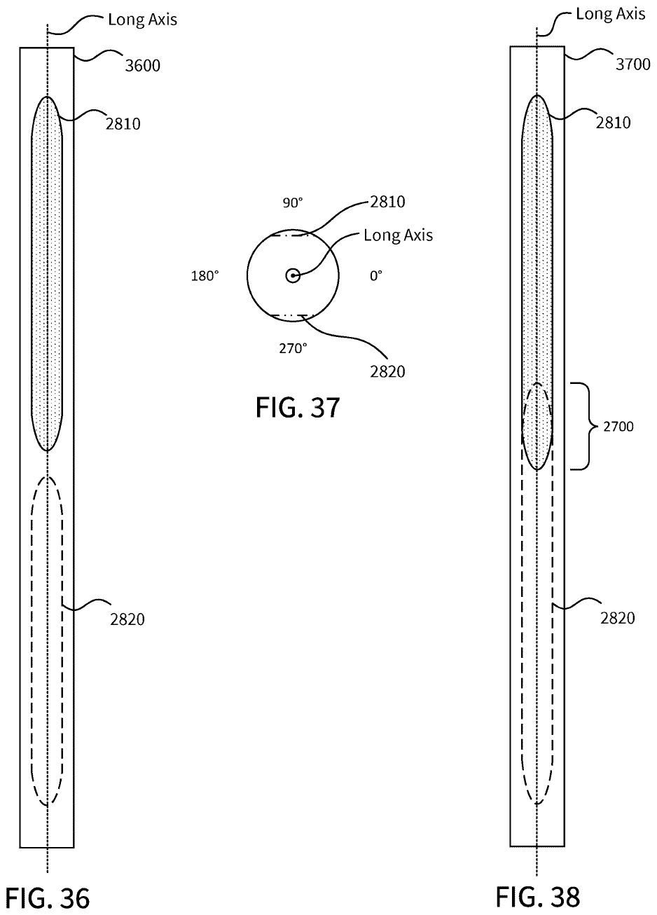

In the above-illustrated configurations, the slots lie on the same line parallel to the longitude of the implant and are not angularly offset from each other (e.g., the slots are centered at approximately the same angle as each other when the implant is view from its top or bottom). In other implementations, one or more of the slots can be arranged such that the slots do not lie on the same line parallel to the longitude of the implant. The slots can be angularly offset with respect to one another, such as by degrees, or some other angle. An example of this is shown in FIGS. 36 and 37.

FIG. 36 illustrates a side view of an implant 3600 having a first slot 2810 and a second slot 2820 (shuttles and other features are omitted for ease of visualization). A long axis extends along the length of the implant 3600. FIG. 37 illustrates a simplified proximal-end view of the implant 3600. As can be seen in this view, the slots 2810, 2820 are arranged at an offset of 180 degrees relative to each other. But in other implementations, the offset can be at least n degrees, where n is an integer between 0 and 180. For example, in the illustrated configuration, the first slot 2810 is rotated 180 degrees about the long axis relative to the second slot 2820.

In the above-illustrated configurations, the slots do not overlap a same plane perpendicular to a length of the implant. However, the implant may be so configured. FIG. 38 illustrates an implant 3700 that is an alternative version of implant 3600. Implant 3700 has slots 2810, 2820 angularly offset with respect to each other and which overlap for an overlap zone 3710. The overlap zone 3710 can be configured in size to permit the shuttles to come together in a way that can cause compression of the bone segments together, such as to improve healing.

In the above-illustrated configurations, the multiple slots of an implant have approximately a same length, but they need not. One slot can be longer than another slot. For example, one slot can be at least x % of the length of another slot, where x is an integer between 0 and 100.

As noted herein, the implants according to various implementations can be used to aid in treatment of a limb length discrepancy and/or a bone defect in a patient's body. That is, the implants disclosed according to various implementations are configured for intramedullary placement in a patient's body. In particular examples, the implant is configured for use in a method of performing an acute shortening and lengthening procedure to the bone segments. In these examples, the implant is configured for bi-directional adjustment to aid in acute shortening and subsequent lengthening, e.g., to improve bone growth during a lengthening procedure. In particular examples, the acute shortening process applies pressure to the point of fusion (aiding in that fusion), prior to the start of the lengthening process. In a particular example, the implant can be used in a lengthening procedure that follows an acute adjustment, e.g., an acute shortening performed by a surgeon on a given bone segment. For example, if a patient has a bone defect (e.g., approximately 10 cm defect), a surgeon may shorten the bone (e.g., by approximately 5 cm) and use the implant to transport (lengthen) that shortened bone to nearly its pre-surgical length (e.g., approximately 5 cm of transport). After transport and docking of the bone, the surgeon may then create a new osteotomy and transport the bone (e.g., using the implant) from another location (e.g., by the additional 5 cm) while the docking site heals. This example approach can allow the surgeon to address specific patient needs in a flexible manner, e.g., by mitigating transport time.

Additional implementations of implants disclosed herein enable a method of performing a trifocal transport procedure. Further implementations of implants disclosed herein enable a method of moving a tibia and/or a femur of a patient. In any case, the implants shown and described according to various implementations enable multi-bone transport, e.g., transport of two or more bone segments with a single intramedullary implant.

In some examples, the implant(s) disclosed herein are configured for extramedullary placement in a patient. In certain cases, a system includes the implant, where the implantable biocompatible housing is configured for intramedullary placement. In these examples, the system further includes an extramedullary plate.

In certain aspects, a method of using an implant herein includes: i) implanting an implant in a patient; coupling a proximal portion of the implant to a proximal bone portion; ii) coupling a distal portion of the implant to a distal bone portion; iii) coupling a first shuttle of the implant to a first bone segment between the proximal and distal bone portions; and iv) coupling a second shuttle of the implant to a second bone segment between the proximal and distal bone portions. In particular implementations, a method further includes: v) causing the first shuttle to move in a first direction at a first rate; and vi) causing the second shuttle to move in a second direction at a second rate. In some examples, the first direction and the second direction are the same direction; and the second rate is greater than the first rate. In additional examples, the first direction and the second direction are opposite, converging directions. In further examples, the first direction and the second direction are opposite, diverging directions. In particular examples, causing the first and second shuttles to move occurs simultaneously. In still further examples, causing the first and second shuttles to move occurs sequentially. In certain cases, implanting an implant in a patient includes disposing the implant in an intramedullary manner. In other cases, implanting an implant in a patient includes disposing the implant in an extramedullary manner.

In additional aspects, an apparatus includes: a means for causing a first bone segment to move in a first direction at a first rate; and a means for causing the second shuttle to move in a second direction at a second rate.

In additional implementations, implants have adjustment dimensions that are defined by a patient adjustment profile for the patient's body. For example, grooves defining adjustment rate and/or extent (groove diameter, pitch, grooves per centimeter, etc), spring constants, gear ratios, etc. can be customized for the patient, in terms of one or more physical characteristic. In a particular example, the pitch of grooves, numbers of grooves per centimeter, spring constants and/or gear ratios are adjustable based on a patient adjustment profile. In such cases, a method of forming an implant (e.g., implant 110 and other implants illustrated herein) includes receiving at least one patient adjustment profile characteristic and assigning a value to at least one of groove pitch, grooves per centimeter, gear ratio, shuttle adjustment extent, etc. in the manufacture of an implant. In certain cases, the patient adjustment profile characteristic includes at least one of: a total translation distance, a total rotation degree, a rate of translation, a rate of rotation, a ratio of translation to rotation, or an adjustment period. The patient adjustment profile characteristics can be defined manually by a healthcare professional. Alternatively, or additionally, surgery planning software can be used to plan the patient adjustment profile characteristics. Once planned, the patient adjustment profile characteristics can be used to manufacture a custom implant (or a customized portion of an implant) for the patient or to assist a surgery team in selecting an implant having a sufficiently close adjustment profile to the determined characteristics.

The implants shown and described herein can be configured for intramedullary placement in a patient, e.g., to aid in treatment of a limb length discrepancy or a bone defect in the patient's body. In certain cases, the implants described and depicted herein can be used in a method of intramedullary adjustment of a patient's bone, e.g., by inserting the implant(s) into the patient's body and by actuating the implant(s) using a controller such as an external control device. In other examples, the implants can be configured for extramedullary placement.

In some examples, the implants described herein can be used as part of a bone transport system with a support member, which may be or include a bone plate configured to be secured to a location on an external surface of the bone in which the implants are implanted. The bone plate may comprise a cortical bone plate. The support member may include one or more holes at its distal end for placement of one or more bone screws. The support member may also include one or more holes at its proximal end for placement of one or more bone screws. The bone screws may be bicortical bone screws and the bone screw may be a unicortical bone screw. Bicortical bone screws may advantageously be used at locations on the bone that are proximal or distal to the adjustable-length implant, while unicortical bone screws may advantageously be used at locations on the bone that are adjacent the implant. The bone screws that are used to secure the support member to the bone may have threaded shafts and tapered, threaded heads that are configured such that the threaded shafts engage with bone material and the tapered threaded heads engage with tapered threaded holes (e.g., the one or more holes) in the support member. The support member maintains the proximal portion and the distal portion of the bone static and stable with respect to each other, thereby optimizing the precision of movement of the transport portion as it is moved in relation to the proximal portion and the distal portion. One or more cerclages may be used to further secure the system in place, for example, to further secure the support member to the bone. In some embodiments, the support member may include consider-ably more holes for placement of bone screws. For example, a portion of the support member configured to be placed at the proximal end of a femur may have three, four, or more holes for placement of bone screws which are configured to be secured into bone and extend into the femoral neck, the greater trochanter, or other portions of the femur, including one or more bone fragments. Additional details regarding how implants described herein can be used with support member are described in U.S. application Ser. No. 16/046,909, previously incorporated herein by reference.

In some examples, the implants described herein can include a distraction loss resistance mechanism, such as described in U.S. application Ser. No. 17/806,552 (filed June 13, 2022), which is incorporated herein by reference in its entirety for any and all purposes. In some examples, the implants described herein can include advanced sealing and retention features, such as is described in PCT Application No. PCT/US2022/031709, filed Jun. 1, 2022, which is incorporated herein by reference in its entirety for any and all purposes. In some examples, the implants described herein can include multi-modal adjustment, such as is described in U.S. Application No. 63/342,921 (filed May 17, 2022), which is incorporated herein by reference in its entirety for any and all purposes.

Any implant described herein can be part of an implantable adjustment system that incorporates an external remote controller (ERC) or other external control device. In certain cases, the ERC can include a magnetic handpiece, a controller (or control box, e.g., with a processor), and a power supply. In additional implementations, the ERC or other external control device can include an interface such as a user interface for enabling a medical professional to interact with the system including implant(s) described herein. Additional details of an ERC and interaction with implants are described in U.S. patent application Ser. No. 16/298,339, previously incorporated by reference herein.

Even further, the implants, associated systems and controllers can include a communication system for connecting devices (e.g., via wireless or hard-wired means), or integral with particular devices (e.g., ERC). The communication system can include a number of hard-wired and/or wireless communication systems, with certain wireless systems configured to communicate over Bluetooth, Bluetooth Low Energy (BLE), radio frequency (RF), Wi-Fi, and/or ultrasound. In additional implementations, the communication system can include an independent subscriber identity module (SIM) assigned to each implant. In further cases, the communication system is configured to communicate wirelessly with a remote control system and/or data gathering/analysis platform, e.g., via a cloud-based communication protocol.

In particular cases, each implant is individually programmable to control an amount of the adjustment of the patient's bone. For example, implants described herein may each include an individually programmable or adjustable component (e.g., programmable controller and/or gear ratio, thread pitch and/or count, etc.) to control the amount of adjustment of the patient's bone. In certain cases, distinct implants in a system can be programmed or otherwise designated to perform distinct adjustments.

In additional cases, the controller(s) described herein includes a smart device (e.g., smart phone, smart watch, tablet, etc.) configured to operate a control platform for adjusting the implants. In these instances, the control platform can include a software application (or “app”) configured to execute or otherwise run at a controller (e.g., ERC) for enabling control of one or more implants. According to certain implementations, the control platform enables control functions for one or more implants from a remote physical location relative to the device. For example, the control platform can enable connection (e.g., network-based and/or cloud-based connection) between a system including the implant(s) described herein and a remote user such as a medical professional.

In all implementations described herein, the implant(s) can further include a feedback system in communication with one or more control devices (e.g., ERC and/or software application running control program). In certain cases, the feedback system provides feedback on a force response to the adjustment of the length of a given adjustment rod and/or rotation of a given adjustment rod. In certain cases, the feedback system includes a sensor onboard the implant, e.g., a sensor that is integrated with or coupled with the housing. Non-limiting examples of sensors can include a load cell, a piezo (piezoelectric) sensor, or an imaging sensor (e.g., optical sensor such as a camera, or an ultrasound sensor). Additional sensors that can be integrated in, or otherwise form part of the feedback system can include position and/or speed sensors (e.g., gyroscope/magnetometer, or inertial measurement unit (IMU)), temperature sensors and/or humidity sensors. In certain cases, the feedback system provides instructions to the controller (e.g., ERC) to modify actuation of a given implant based on the feedback on the force response.

In still further implementations, the sensor(s) in the feedback system described herein can be configured to provide data about a load exerted on an adjustment element, and/or a load exerted by the adjustment element on the patient's bone. The sensor(s) can also provide data about a tensile load between the implants and bone. In certain implementations, both torque and compression data are recorded by sensor(s) and provided to the feedback system for analysis and/or action (e.g., to adjust adjustment instructions). It is understood that torque and/or compression data detected by sensors, can represent an inferred or correlated indicator of the torque and/or compression applied to a device or component not physically in contact with the sensor. For example, the sensor on an instrument can be configured to detect torque at the instrument, while that torque is being translated to a driven element in contact with the distal end of the instrument. Similarly, the sensor on an instrument can detect compression at the instrument, while that compression is being translated to an external component, e.g., a driven element.

In additional implementations, one or more device components described herein, e.g., driving elements in implants, can be communicatively coupled with a navigation system that is configured to detect a position of the instrument(s). In one example, the control unit (e.g., ERC) can include or otherwise communicate with a navigation system in order to provide navigation information about a position of instruments. For example, the navigation system can include an optical tracking system such as a camera or laser-based tracking system, a Global Positioning System (GPS), an inertial measurement unit (IMU), an ultrasound based measurement system, other kinds of position systems, or combinations thereof. In certain cases, the navigation system is configured to determine a distance moved by the instrument when the instrument changes position, which the navigation system communicates to the control unit (e.g., for processing by the feedback system). One or more components of a navigation system can be located within or otherwise integrated with a housing that is mounted to or otherwise coupled with one or more of the device components.

In certain cases, the feedback system, or functions thereof, can be integrated into a control unit and/or a controller as described herein. In particular cases, the feedback system is part of a software application and is configured to determine what, if any, force adjustment should be made at a given implant based on the force feedback. In some examples, the feedback system includes a model that correlates force response and force applied during adjustment of the length of an implant. The model can be based at least in part on historical data from a set of implants in distinct bone fixation devices, e.g., similar to implant(s) described herein. According to various implementations, the model can be updated periodically, or on a continuous basis, to provide additional data about force response as compared to force applied in one or more implants. In certain cases, a version of the model can be downloaded or otherwise stored locally at one or more control units and/or controllers and periodically updated, e.g., via a cloud-based or other network-based software update. This approach can reduce the computational and/or storage requirements at control unit(s) and controller(s) that may be local to the implant(s).

In additional implementations, the feedback system is configured to provide post-operative data, post-adjustment data, and analysis of alignment procedure and/or device usage, e.g., to enhance future procedures and/or diagnose inefficiencies in a past procedure. In certain implementations, the feedback system is configured to update the control instructions for control unit(s) based on identified inefficiencies or errors in adjustment quantities (e.g., lengthening, rotation) and/or device usage during/after a given procedure. In particular implementations, the feedback system includes a logic engine configured to modify instructions iteratively, e.g., on a procedure-by-procedure or patient-by-patient basis.

Various additional aspects of the disclosure can include a method of intramedullary adjustment of a patient's bone using the implant(s) described herein. Using FIGS. 2 and 3 strictly for the simplicity of illustration, the method can include adjusting a patient's bone using an intramedullary implant such as implant 110 by: (i) coupling the implant 110 to the patient's bone (e.g., via bone screws or other fasteners at holes); and (ii) actuating adjustment of the length of the implant 110 with an external control device (e.g., ERC or other remote controller). In certain cases, after adjusting the patient's bone, a method can further include: (iii) decoupling the implant from the patient's bone (e.g., via bone screws or other fasteners at holes).

In certain cases, a method can include imaging a bone connected with the implant(s) described and illustrated herein. For example, a method can include: (I) coupling or decoupling an implant (e.g., implant 110) with a patient's bone, and (II) imaging the bone with MRI and/or X-ray imaging after the coupling or decoupling. After imaging, the method can further include: (III) either (a) adjusting an already coupled implant (e.g., implant 110) or (b) decoupling the already coupled implant (e.g., implant 110) based on feedback from the imaging process.

As noted herein, the implants and associated methods described herein enable multi-bone adjustment, which can reduce time and complications associated with bone adjustment procedures. The various disclosed implementations can improve patient outcomes when compared with conventional implantable adjusters, for example, increasing adaptability in adjusting bone positioning, enhancing both intraoperative and postoperative engagement with the device. When compared with conventional approaches, the multi-bone (e.g., two or more bone) adjustment implants described according to various implementations provide an efficient and simplified mechanism for bone adjustment. The implants described according to various implementations can also reduce health risks for patients when compared with conventional approaches, for example, allowing a single implant to perform functions conventionally performed by multiple implantable devices (with associated implant procedures).

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as, special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultra-sonic welding, bonding). In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

Commonly labeled components in the FIGURES are considered to be substantially equivalent components for the purposes of illustration, and redundant discussion of those components is omitted for clarity.

While inventive features described herein have been described in terms of preferred embodiments for achieving the objectives, it will be appreciated by those skilled in the art that variations may be accomplished in view of these teachings without deviating from the spirit or scope of the invention. Also, while this invention has been described according to a preferred use in spinal applications, it will be appreciated that it may be applied to various other uses desiring surgical fixation, for example, the fixation of long bones.

Various example embodiments of devices (e.g., implants) and techniques for moving bone in a patient's body are described herein. In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The implants and related systems, program products and methods described herein boast a variety of inventive features and components that warrant patent protection, both individually and in combination.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

A number of implementations have been described. Nevertheless, it will be understood that additional modifications may be made without departing from the scope of the inventive concepts described herein, and, accordingly, other implementations are within the scope of the following claims.

We claim:

1. An implant for moving a plurality of separate bone segments between bone end-segments in a patient's body, the implant comprising:
an implantable biocompatible housing defining:
a proximal hole for receiving a bone anchor coupled to a proximal bone end-segment; and
a distal hole for receiving a bone anchor coupled to a distal bone end-segment;
a first shuttle coupled with the housing and defining a first set of one or more holes for receiving a bone anchor coupled to a first bone segment in the plurality of bone segments;
a second shuttle coupled with the housing and defining a second set of one or more holes for receiving a bone anchor coupled to a second bone segment in the plurality of bone segments; and
a drive system disposed at least partially within the housing and configured to translate the first and second shuttles relative to the housing to move the first and second bone segments, respectively.

2. The implant of claim 1, wherein the first shuttle includes a first translatable rack mounted in the housing and the second shuttle includes a second translatable rack mounted in the housing.

3. The implant of claim 2, wherein the drive system comprises at least one gear, the implant further comprising:
a first rail coupled with the first shuttle and the at least one gear; and
a second rail coupled with the second shuttle and the at least one gear, wherein movement of the first rail or the second rail translates movement to the other of the first rail or the second rail.

4. The implant of claim 3, wherein the drive system directly drives the first shuttle or the second shuttle and indirectly drives the other of the first shuttle or the second shuttle.

5. The implant of claim 4, wherein the drive system includes a magnetic driver configured to be driven by an actuator in an external control device from a location external to the patient's body.

6. The implant of claim 3, wherein the at least one gear includes a first gear and a second gear, wherein the first gear and the second gear are coupled by a common axis.

7. The implant of claim 6, wherein the first gear and the second gear have distinct gear ratios.

8. The implant of claim 1, wherein the drive system includes at least one of:
an electric driver configured to be driven by an actuator in an external control device from a location external to the patient's body, or
an ultrasound powered driver configured to be driven by an actuator in an external control device from a location external to the patient's body.

9. The implant of claim 1, further comprising:
a lead screw coupled with the drive system and the first shuttle;
a first rack coupled with the first shuttle and the second shuttle;
a second rack coupled with the second shuttle and the housing; and
a rack gear coupled with the second shuttle, the first rack and the second rack.

10. The implant of claim 9, further comprising a pinion gear between the rack gear and the second shuttle to modify a gear ratio of the second shuttle relative to the rack gear.

11. The implant of claim 9, wherein the first rack is fixed to the first shuttle, the second rack is fixed to the housing, and wherein the second shuttle is configured to move relative to the first rack and the second rack.

12. The implant of claim 11, wherein the drive system is configured to drive translation of the lead screw, the first shuttle and the first rack, and wherein translation of the first rack engages the rack gear at a first rate and causes translation of the second shuttle at a second, distinct rate.

13. The implant of claim 1, wherein the drive system includes a first driver coupled with the first shuttle and a second driver coupled with the second shuttle, wherein the first shuttle and the second shuttle are axially interposed between the first driver and the second driver.

14. The implant of claim 13, wherein the first driver and the second driver each include a magnetic driver configured to be driven by an actuator in at least one external control device from a location external to the patient's body.

15. The implant of claim 14, wherein the first driver and the second driver are axially separated to mitigate magnetic field interference during actuation by the external control device.

16. The implant of claim 13, wherein the first driver and the second driver are configured to be actuated for at least one of converging or series transport of the plurality of bone segments.

17. The implant of claim 1, wherein the drive system includes a single driver coupled with both the first shuttle and the second shuttle.

18. The implant of claim 17, wherein the single driver includes a magnetic driver coupled with two distinct gearboxes, wherein actuation of the magnetic driver causes either symmetrical translation of the first shuttle and the second shuttle or asymmetrical translation of the first shuttle with respect to the second shuttle.

19. The implant of claim 1, wherein the housing includes a first sub-housing encompassing the first shuttle and a second sub-housing encompassing the second shuttle.

20. The implant of claim 19, further comprising a distraction rod spanning axially between the first sub-housing and the second sub-housing.

21. The implant of claim 20, further comprising:
a first lead screw in the first sub-housing that is coupled with a first portion of the distraction rod; and
a second lead screw in the second sub-housing that is coupled with a second portion of the distraction rod,
wherein the drive system includes: a first driver in the first sub-housing that is coupled with the first lead screw; and a second driver in the second sub-housing that is coupled with the second lead screw,
wherein the first driver and the second driver are separately controllable to translate the plurality of bone segments.

22. The implant of claim 21, wherein the first lead screw has a first thread orientation and the second lead screw has a second, distinct thread orientation.

23. The implant of claim 21, wherein the first driver and the second driver enable a multi-stage lengthening of the plurality of bone segments.

24. The implant of claim 1, further comprising a distraction rod within the housing, wherein the first shuttle is internal to the distraction rod and the second shuttle is configured to be driven by the distraction rod.

25. The implant of claim 24, wherein the drive system is coupled with the distraction rod for driving the distraction rod.

26. The implant of claim 24, wherein the first shuttle includes a first threaded coupling with the distraction rod.

27. The implant of claim 26, wherein the drive system includes a lead screw with a second threaded coupling to the distraction rod, wherein the first threaded coupling and the second threaded coupling have distinct thread pitches.

28. The implant of claim 27, wherein the first threaded coupling has thread pitch that is one-half a thread pitch of the second threaded coupling.

29. The implant of claim 1, further comprising:
a distraction rod coupled with the drive system and the first shuttle; and
a cable assembly coupled with the drive system and the second shuttle.

30. The implant of claim 29, wherein the cable assembly includes a winder coupled to the drive system and a cable connected with the second shuttle and the winder, wherein actuation of the drive system causes translation of the distraction rod and the first shuttle and winding of the winder to translate the second shuttle.

31. The implant of claim 30, further comprising a spring coupled with the second shuttle to provide counter-tension on the second shuttle during the winding of the winder.

32. The implant of claim 1, further comprising a lead screw coupled with the drive system, wherein each of the first shuttle and the second shuttle is directly coupled with the lead screw.

33. The implant of claim 32, wherein the housing includes an axially extending slot enabling direct coupling of the bone anchors to each of the first and second sets of holes.

34. The implant of claim 33, wherein the first shuttle and the second shuttle are configured to move axially along the slot.

35. The implant of claim 34, wherein the lead screw includes a first threaded coupling with the first shuttle having a first thread pitch and a second threaded coupling with the second shuttle having a second, distinct thread pitch.

36. The implant of claim 1, further comprising:
a lead screw coupled with the drive system;
at least one band coupled with the lead screw and each of the first shuttle and the second shuttle; and
a reel coupled with the at least one band.

37. The implant of claim 36, further comprising a tether on the lead screw that is coupled with the at least one band.

38. The implant of claim 36, wherein actuation of the drive system causes the lead screw to travel within the housing and cause movement of each of the first shuttle and the second shuttle via the at least one band.

39. The implant of claim 36, wherein the housing includes an axially extending slot enabling direct coupling of the bone anchors to each of the first and second sets of holes.

40. The implant of claim 39, wherein the first shuttle and the second shuttle are configured to move axially along the slot.

41. The implant of claim 36, wherein the reel is axially interposed between the first shuttle and the second shuttle.

42. The implant of claim 1, further comprising:
a lead screw coupled with the drive system and the first shuttle; and
a spring system coupled with the first shuttle and the second shuttle, wherein the spring system is configured to provide a translation differential between the first shuttle and the second shuttle when driven by the lead screw.

43. The implant of claim 42, wherein a portion of the spring system is axially interposed between the first shuttle and the second shuttle.

44. The implant of claim 43, wherein the spring system includes a first spring and a second spring with distinct spring constants, wherein the first spring is axially interposed between the first shuttle and the second shuttle, and wherein the second spring is axially interposed between the second shuttle and a distal end of the implant.

45. The implant of claim 42, wherein when driven by the drive system and the lead screw, the first shuttle translates at a first rate and the second shuttle translates at a second, distinct rate.

46. The implant of claim 45, wherein the second rate is at least one-half of the first rate.

47. The implant of claim 42, wherein the spring system self-centers the first shuttle and the second shuttle relative to the housing in response to driving by the drive system.

48. The implant of claim 1, wherein the first shuttle includes the drive system having a magnetic driver, a gearbox coupled with the magnetic driver, and a lead screw coupled with the gearbox, wherein a portion of the lead screw protrudes axially from the first shuttle.

49. The implant of claim 48, wherein the magnetic driver is configured to be driven by an actuator in an external control device from a location external to the patient's body, and wherein actuation of the magnetic driver causes the magnetic driver to translate axially within the housing.

50. The implant of claim 48, wherein the second shuttle is coupled with the lead screw and is configured to translate with movement of the lead screw.

51. The implant of claim 50, wherein the magnetic driver includes a threaded coupler for modifying a rate of translation of the first shuttle relative to the second shuttle.

52. The implant of claim 1, further comprising a distraction rod housing the drive system, wherein the first shuttle is coupled with the drive system.

53. The implant of claim 52, wherein the drive system includes a magnetic driver and a gearbox for driving a lead screw, and wherein the first shuttle includes an anti-rotation feature for preventing rotation of the first shuttle during rotation of the lead screw.

54. The implant of claim 53, wherein the second shuttle is coupled with the lead screw and is configured to translate proportionally with the lead screw.

55. The implant of claim 54, wherein the first shuttle is configured to translate disproportionally with the lead screw.

56. The implant of claim 1, wherein the first shuttle and the second shuttle each include a housing and wherein the set of one or more holes extend at least partially through the housing.

57. The implant of claim 1, wherein the first shuttle and the second shuttle are configured to translate relative to the housing to move the first bone segment and the second bone segment according to a patient adjustment profile for the patient's body.

58. The implant of claim 1, wherein the drive system is configured to cause the first and second shuttles to:
move in a same direction at different rates from each other, or
move in opposite, converging or diverging directions.

59. The implant of claim 1, wherein the implant is configured for intramedullary placement in a patient.

60. The implant of claim 1, wherein the implant is configured to aid in treatment of a limb length discrepancy or a bone defect in the patient's body.

61. A method of performing an acute shortening and lengthening procedure to the plurality of bone segments with the implant of claim 1.

62. A method of performing a trifocal transport procedure with the implant of claim 1.

63. A method of moving at least one of a tibia or a femur of the patient with the implant of claim 1.

64. The implant of claim 1, wherein the implant is configured for extramedullary placement in a patient.

65. A system comprising:
the implant of claim 1, wherein the implantable biocompatible housing is configured for intramedullary placement; and
an extramedullary plate.

66. A method comprising:
implanting an implant in a patient;
coupling a proximal portion of the implant to a proximal bone portion;
coupling a distal portion of the implant to a distal bone portion;
coupling a first shuttle of the implant to a first bone segment between the proximal and distal bone portions; and
coupling a second shuttle of the implant to a second bone segment between the proximal and distal bone portions.

67. The method of claim 66, further comprising:
causing the first shuttle to move in a first direction at a first rate; and
causing the second shuttle to move in a second direction at a second rate.

68. The method of claim 67,
wherein the first direction and the second direction are the same direction; and
wherein the second rate is greater than the first rate.

69. The method of claim 67, wherein the first direction and the second direction are opposite, converging directions.

70. The method of claim 67, wherein the first direction and the second direction are opposite, diverging directions.

71. The method of claim 67, wherein the causing of the first and second shuttles to move occurs simultaneously.

72. The method of claim 67, wherein the causing of the first and second shuttles to move occurs sequentially.

73. The method of claim 66,
wherein implanting an implant in a patient includes disposing the implant in an intramedullary manner.

74. The method of claim 66, wherein implanting an implant in a patient includes disposing the implant in an extramedullary manner.

75. An apparatus comprising:
a means for causing a first bone segment to move in a first direction at a first rate; and
a means for causing the second shuttle to move in a second direction at a second rate.

76. An implant for moving a plurality of separate bone segments between bone end-segments in a patient's body, the implant comprising:
an implantable biocompatible housing defining:
a first slot;
a second slot; and
a bridge separating the first slot and the second slot;
a first shuttle coupled with the housing and defining a first set of one or more holes for receiving a bone anchor coupled to a first bone segment in the plurality of bone segments;
a second shuttle coupled with the housing and defining a second set of one or more holes for receiving a bone anchor coupled to a second bone segment in the plurality of bone segments;
a drive system configured to translate the first and second shuttles relative to the housing to move the first and second bone segments, respectively.

77. The implant of claim 76,
wherein the first shuttle is disposed within the first slot; and
wherein the second shuttle is disposed within the second slot.

78. The implant of claim 77, wherein the drive system is configured to cause the first and second shuttles to move towards each other.

79. The implant of claim 76, wherein the first shuttle and the second shuttle are disposed within the first slot.

80. The implant of claim 79, wherein the drive system is configured to cause the first and second shuttles to move in a same direction.

81. The implant of claim 76, wherein the first slot and the second slot are different lengths.

82. The implant of claim 76, wherein the first slot and the second slot extend along respective parallel, non-overlapping lines.

83. The implant of claim 76, wherein the first slot is rotated 180 degrees about the long axis of the implant relative to the second slot.

84. The implant of claim 83, wherein a plane perpendicular to the long axis of the implant intersects both the first slot and the second slot.

85. An implant for moving a plurality of separate bone segments between bone end-segments in a patient's body, the implant comprising:
an implantable biocompatible housing defining:
a first slot;
a second slot;
a third slot;
a fourth slot;
a first bridge separating the first slot and the second slot;
a second bridge separating the second slot and third slot; and
a third bridge separating the third slot and the fourth slot;
a first shuttle coupled with the housing and defining a first set of one or more holes for receiving a bone anchor coupled to a first bone segment in the plurality of bone segments;
a second shuttle coupled with the housing and defining a second set of one or more holes for receiving a bone anchor coupled to a second bone segment in the plurality of bone segments; and
a drive system configured to translate the first and second shuttles relative to the housing to move the first and second bone segments, respectively.

86. The implant of claim 85,
wherein the first shuttle is disposed within the first slot;
wherein the second shuttle is disposed within the fourth slot; and
wherein the second and third slots are between the first and fourth slots.
